(19) United States
(12) Patent Application Publication
(10) Pub. No.: US 2021/0379584 A1
(43) Pub. Date: Dec. 9, 2021

(54) Multiplexed Sample Plate

(71) Applicant: Dynex Technologies, Inc., Chantilly, VA (US)

(72) Inventors: Deval A. LASHKARI, Moraga, CA (US); James BAILEY, Arlington, VA (US)

(21) Appl. No.: 17/260,705

(22) PCT Filed: Jul. 2, 2019
(86) PCT No.: PCT/GB2019/051873

§ 371 Date: Jan. 15, 2021

Related U.S. Application Data
(60) Provisional application No. 62/700,598, filed on Jul. 19, 2018.

(51) Int. Cl. B01L 3/00

(52) U.S. Cl. CPC B01L 3/50855; B01L 2200/12; B01L 2200/0668; B01L 2300/0829

(57) ABSTRACT

A multiplexed sample plate comprising a sample well is disclosed. A plurality of substantially cylindrical reagent bead 2500 are inserted in use within a hole or aperture of the sample well. The substantially cylindrical reagent beads are positioned so as not to protrude beyond an upper surface of the base portion.

### CROSS-REFERENCE TO RELATED APPLICATIONS

None

### BACKGROUND TO THE INVENTION

The present invention relates to a sample plate, a multiplexed sample plate, a method of assaying for one or more analytes of interest, an automated apparatus, a kit for performing Enzyme Linked ImmunoSorbent Assay procedures, a kit for performing nucleic acid probe procedures, a method of manufacturing a sample plate, a method of manufacturing substantially or generally cylindrical reagent beads, plugs or inserts and a method of multiplex analysis.

A sample plate or multiplexed sample plate is disclosed which may be used to carry out diagnostic testing such as Enzyme Linked ImmunoSorbent Assay (“ELISA”) procedures or other immunoassay procedures. Alternatively, the sample plate or multiplexed sample plate may be used to carry out testing for DNA or RNA sequences.

Immunoassay procedures are a preferred way of testing biological products. These procedures exploit the ability of antibodies produced by the body to recognise and combine with specific antigens which may, for example, be associated with foreign bodies such as bacteria or viruses, or with other body products such as hormones. Once a specific antigen-antibody combination has occurred it can be detected using chromogenic, fluorescent or chemiluminescent materials or less preferably by using radioactive substances. Radioactive substances are less preferred due to environmental and safety concerns regarding their handling, storage and disposal. The same principles can be used to detect or determine any materials which can form specific binding pairs, for example using lectins, rheumatoid factor, protein A or nucleic acids as one of the binding partners.

ELISA is a particularly preferred form of immunoassay procedure wherein one member of the binding pair is linked to an insoluble carrier surface (“the solid phase”) such as a sample vessel, and after reaction the bound pair is detected by use of a further specific binding agent conjugated to an enzyme (“the conjugate”). The procedures for ELISA are well known in the art and have been in use for both research and commercial purposes for many years. Numerous books and review articles describe the theory and practice of immunoassays. Advice is given, for example, on the characteristics and choice of solid phases for capture assays, on methods and reagents for coating solid phases with capture components, on the nature and choice of labels, and on methods for labelling components. An example of a standard textbook is “ELISA and Other Solid Phase Immunoassays, Theoretical and Practical Aspects”, Editors D. M. Kemeny & S. J. Challacombe, published by John Wiley, 1988. Such advice may also be applied to assays for other specific binding pairs.

In the most common type of ELISA, the solid phase is coated with a member of the binding pair. An aliquot of the specimen to be examined is incubated with the solid coated solid phase and any analyte that may be present is captured onto the solid phase. After washing to remove residual specimen and any interfering materials it may contain, a second binding agent, specific for the analyte and conjugated to an enzyme is added to the solid phase. During a second incubation any analyte captured onto the solid phase will combine with the conjugate. After a second washing to remove any unbound conjugate, a chromogenic substrate for the enzyme is added to the solid phase. Any enzyme present will begin to convert the substrate to a chromophoric product. After a specified time the amount of product formed may be measured using a spectrophotometer, either directly or after stopping the reaction.

It will be realised that the above is an outline description of a general procedure for a bioassay and that many variants are known in the art including fluorogenic and luminogenic substrates for ELISA, direct labelling of the second member of the binding pair with a fluorescent or luminescent molecule (in which case the procedure is not called an ELISA but the process steps are very similar) and nucleic acids or other specific pairing agents instead of antibodies as the binding agent. However, all assays require that fluid samples, e.g. blood, serum, urine, etc., are aspirated from a sample tube and are then dispensed into a solid phase. Samples may be diluted prior to being dispensed into the solid phase or they may be dispensed into deep well microplates, diluted in situ and then the diluted analyte may be transferred to the functional solid phase.

The most common type of solid phase is a standard sample vessel known as a microplate which can be stored easily and which may be used with a variety of biological specimens. Microplates have been available commercially since the 1960s and are made from e.g. polystyrene, PVC, Perspex or Lucite and measure approximately 5 inches (12.7 cm) in length, 3.3 inches (8.5 cm) in width, and 0.55 inches (1.4 cm) in depth. Microplates made from polystyrene are particularly preferred on account of polystyrene's enhanced optical clarity which assists visual interpretation of the results of any reaction. Polystyrene microplates are also compact, lightweight and easily washable. Microplates manufactured by the Applicants are sold under the name “MICROTITRE”®. Known microplates comprise 96 wells (also commonly known as “microwells”) which are symmetrically arranged in an 8×12 array. Microwells typically have a maximum volume capacity of approximately 350 μl. However, normally only 10-200 μl of fluid is dispensed into a microwell. In some arrangements of the microplate the microwells may be arranged in strips of 8 or 12 wells that can be moved and combined in a carrier to give a complete plate having conventional dimensions.

Positive and negative controls are generally supplied with commercial kits and are used for quality control and to provide a relative cut-off. After reading the processed microplate, the results of the controls are checked against the manufacturer's validated values to ensure that the analysis has operated correctly and then the value is used to distinguish positive from negative specimens and a cut-off value is calculated. Standards are usually provided for quantitative assays and are used to build a standard curve from which the concentration of analyte in a specimen may be interpolated.

It will be recognised that the ELISA procedure as outlined above involves multiple steps including pipetting, incubation, washing, transferring microplates between activities, reading and data analysis. In recent years systems have been developed which automate the steps (or “phases”) involved in the ELISA procedures such as sample distribution, dilution, incubation at specific temperatures, washing, enzyme conjugate addition, reagent addition, reaction stopping and the analysis of results. The pipette mechanism used to aspirate and dispense fluid samples uses disposable tips which are ejected after being used so as to prevent cross-contamination of patients' samples. Multiple instrumental controls are in place to ensure that appropriate volumes, times, wavelengths and temperatures are employed, data transfer and analysis is fully validated and monitored. Automated immunoassay apparatus for carrying out ELISA procedures are now widely used in laboratories of e.g. pharmaceutical companies, veterinary and botanical laboratories, hospitals and universities for in-vitro diagnostic applications such as testing for diseases and infection, and for assisting in the production of new vaccines and drugs.

ELISA kits are commercially available which consist of microplates having microwells which have been coated by the manufacturer with a specific antibody (or antigen). For example, in the case of a hepatitis B antigen diagnostic kit, the kit manufacturer will dispense anti-hepatitis B antibodies which have been suspended in a fluid into the microwells of a microplate. The microplate is then incubated for a period of time, during which time the antibodies adhere to the walls of the microwells up to the fluid fill level (typically about half the maximum fluid capacity of the microwell). The microwells are then washed leaving a microplate having microwells whose walls are uniformly covered with anti-hepatitis B antibodies up to the fluid fill level.

A testing laboratory will receive a number of sample tubes containing, for example, body fluid from a number of patients. A specified amount of fluid is then aspirated out of the sample tube using a pipette mechanism and is then dispensed into one or more microwells of a microplate which has been previously prepared by the manufacturer as discussed above. If it is desired to test a patient for a number of different diseases then fluid from the patient must be dispensed into a number of separate microplates, each coated by the manufacturer with a different binding agent. Each microplate must then be processed separately to detect the presence of a different disease. It will be understood that to analyse several different analytes requires a multiplicity of microplates and transfer of aliquots of the same specimen to the different microplates. This leads to large numbers of processing steps, incubators and washing stations that can cope with many microplates virtually simultaneously. In automated systems this requires instruments to have multiple incubators and complex programming is required to avoid clashes between microplates with different requirements. For manual operation either several technicians are required or the throughput of specimens is slow. It is possible to combine strips of differently coated microwells into a single carrier, add aliquots of a single specimen to the different types of well and then perform the ELISA in this combined microplate. Constraints on assay development, however, make this combination difficult to achieve and it is known that for users to combine strips in this fashion can lead to errors of assignment of result, while manufacture of microplates with several different coatings in different microwells presents difficulties in terms of quality control.

Conventional ELISA techniques have concentrated upon performing the same single test upon a plurality of patient samples per microplate or in detecting the presence of one or more of a multiplicity of analytes in those patients without distinguishing which of the possible analytes is actually present. For example, it is commonplace to determine in a single microwell whether a patient has antibodies to HIV-1 or HIV-2, or HIV-1 or -2 antigens, without determining which analyte is present and similarly for HCV antibodies and antigens.

However, a new generation of assays are being developed which enable multiplexing to be performed. Multiplexing enables multiple different tests to be performed simultaneously upon the same patient sample.

A recent approach to multiplexing is to provide a microplate comprising 96 sample wells wherein an array of different capture antibodies is disposed in each sample well. The array comprises an array of 20 nl spots each having a diameter of 350 μm. The spots are arranged with a pitch spacing of 650 μm. Each spot corresponds with a different capture antibody.

Multiplexing enables a greater number of data points and more information per assay to be obtained compared with conventional ELISA techniques wherein each sample plate tests for a single analyte of interest. The ability to be able to combine multiple separate tests into the same assay can lead to considerable time and cost savings. Multiplexing also enables the overall footprint of the automated apparatus to be reduced.

Although there are many advantageous aspects to current known ELISA techniques and to the new multiplex techniques which are currently being developed, it is nonetheless desired to provide a sample plate and associated automated apparatus which has an improved format and which provides a greater flexibility than state of the art ELISA arrangements.

In addition to ELISA procedures it is also known to use a hybridization probe to test for the presence of DNA or RNA sequences. A hybridization probe typically comprises a fragment of DNA or RNA which is used to detect the presence of nucleotide sequences which are complementary to the DNA or RNA sequence on the probe. The hybridization probe hybridizes to single-stranded nucleic acid (e.g. DNA or RNA) whose base sequence allows pairing due to complementarity between the hybridization probe and the sample being analysed. The hybridization probe may be tagged or labelled with a molecular marker such as a radioactive or more preferably a fluorescent molecule. The probes are inactive until hybridization at which point there is a conformational change and the molecule complex becomes active and will then fluoresce (which can be detected under UV light) DNA sequences or RNA transcripts which have a moderate to high sequence similarity to the probe are then detected by visualising the probe under UV light.

An assay device and assembly for detecting an analyte in a liquid sample is disclosed in U.S. Pat. No. 5,620,853 (Chiron Corporation). The assay device comprises a moulded well comprising fingers which protrude up from the bottom of the well and into which a reagent bead is dispensed. The reagent bead is captured in the fingers but can still move up and down within the finger height. The assay device is arranged to expose the reagent bead to as much fluid flow as possible and to rely upon signal from the underside of the reagent bead to produce results.

There are a number of problems with the arrangement disclosed in U.S. Pat. No. 5,620,853 (Chiron Corporation). Firstly, since the reagent beads are free to move up and down within the finger height then it is possible that a reagent bead may become stuck at an undesired height during a processing or reading step. In particular, the design of the well is relatively intricate and complex and any movement of, or damage to, the fingers could result in a reagent bead becoming stuck at an undesired height. The fingers also protrude from the base which makes them susceptible to damage particularly during pipetting and washing stages. If a reagent bead does become stuck at an undesired height within the fingers then this is highly likely to have an adverse effect upon the accuracy of the testing procedures.

Secondly, the design of the well with fingers which are arranged to receive a single reagent bead is such that fluid is pipetted next to the bead and the bead is covered by the rising fluid in the well. The single wells need approximately 300 μl of fluid. U.S. Pat. No. 5,620,853 (Chiron Corporation) also discloses an arrangement wherein multiple wells are in fluid communication with each other. For the multi-well arrangement, each well will need approximately 300 μl of fluid. It will be apparent, therefore, that the multi-well arrangement requires an excessive amount of fluid to be dispensed relative to conventional systems.

Thirdly, the arrangement of fingers reduces the maximum packing density of wells for a given size sample plate so that relatively few tests can be performed on a given sample plate.

Fourthly, the multi-well arrangement disclosed in U.S. Pat. No. 5,620,853 (Chiron Corporation) is particularly prone to crosstalk.

Fifthly, the arrangement disclosed in U.S. Pat. No. 5,620,853 (Chiron Corporation) is such that when a single bead is used then the homogeneity of the fluid is only affected by the protruding fingers. There are likely to be regions of the well which will trap unmixed fluid. The multi-well arrangement also suffers from the serious problem that any fluid required to go over all beads has to pass through a tortuous path to get from one well to another. This will cause serious problems in terms of fluid mixing and bead to bead repeatability. The single well arrangement is completely different to the in-line multi-well arrangement disclosed in U.S. Pat. No. 5,620,853 (Chiron Corporation) and the two different arrangements would therefore have quite different fluid characteristics. This is likely to result in different fluid behaviours depending upon the arrangement used and hence there is likely to be significant variation in results depending upon whether a single well or a multi-well format was used. Although in theory the two different arrangements could be validated independently, this would result in increased cost and reduced throughput.

Finally, the sample well disclosed in U.S. Pat. No. 5,620,853 (Chiron Corporation) is relatively complex to manufacture and is likely to suffer from unreliability issues during manufacture. The long thin fingers are difficult to form by moulding and would be prone to damage during manufacture or during use. The fingers also have a feature at the top which in a mould tool would be an undercut. When the part is ejected off the tool the fingers must bend for the feature to get past the tool material. Such a manufacturing process is generally undesirable due to unreliability issues. Furthermore, any change in the process parameters is likely to affect the ability to release the part from the tool and leave the part intact to the correct mechanical tolerances. The position of the fingers relative to each other is critical to allow the reagent bead to move up and down correctly and also to ensure that the reagent bead does not come out of the top of the fingers. This would be very difficult, in practice, to control in a mass production environment. It is also noted that the design of the single bead arrangement is completely different to the design of the multi-well arrangement. As a result, completely different tool designs would be required which again would greatly increase the complexity of manufacture. In a high volume manufacturing environment the combination of the design features and quality assurance concerns would make the sample plates excessively expensive to produce.

US 2009/0069200 (Yu) discloses a system for preparing arrays of biomolecules. According to the arrangement disclosed in US 2009/0069200 (Yu) spherical beads are arranged within subwells which have a square cross-section. The spherical beads do not form a circumferential seal with the wall of the subwell and as a result fluid passes up from the bottom of the subwell, past the beads and over the top of the beads so that the beads are fully submerged or immersed. There are a number of problems with such an arrangement which are discussed in more detail later in the present application.

A multiplexed sample plate is known comprising a plurality sample wells, wherein each sample well comprise a base portion and wherein a plurality of open through holes are provided in the base portion. A spherical reagent bead or microsphere is substantially retained or secured, in use, within each through hole so as to form a substantially fluid-tight circumferential seal with a wall of the base portion which defines the through hole. Each spherical reagent bead protrudes above the base portion into the sample well.

One problem with the known sample plate which utilises spherical reagent beads is that the known arrangement suffers from the problem of crosstalk between neighbouring beads when the luminosity of the reagent beads is being determined at a plate reading stage. This problem is discussed in more detail below.

It is desired to provide an improved sample plate or multiplexed sample plate which does not suffer from the problem of crosstalk when being read.

### SUMMARY OF THE INVENTION

According to an aspect there is provided a sample plate or multiplexed sample plate comprising one or more sample wells, wherein one or more of the sample wells comprise:

a base portion having an upper surface which forms a bottom portion of the sample well; and

one or more holes or apertures provided in the base portion;

wherein one or more non-spherical reagent beads, plugs or inserts are substantially retained or secured, in use, within the one or more holes or apertures so as to form a substantially fluid-tight circumferential seal with a wall of the base portion which defines the hole or aperture.

An upper surface of the one or more reagent beads, plugs or inserts preferably does not substantially protrude above or beyond the upper surface of the base portion.

Other less preferred embodiments are contemplated wherein the one or more non-spherical reagent beads, plugs or inserts may protrude, for example, ≤1 mm, ≤2 mm, ≤3 mm, ≤4 mm or ≤5 mm above and beyond the upper surface of the base portion.

According to another embodiment the one or more non-spherical reagent beads, plugs or inserts may be recessed, for example, ≤1 mm, ≤2 mm, ≤3 mm, ≤4 mm or ≤5 mm below the upper surface of the base portion.

The preferred sample plate or multiplexed sample plate is particularly advantageous in that crosstalk between reagent beads, plugs or inserts when the reagent beads, plugs or inserts located in the sample plate or multiplexed sample plate are read by a plate reader is substantially reduced or eliminated. In particular, the use of a crosstalk correction algorithm to correct for the effects of crosstalk between neighbouring reagent beads, plugs or inserts in a sample well when the sample well is being read by a plate reader to determine the luminosity of the reagent beads, plugs or inserts can be avoided.

It will be understood that sample plates, multiplexed sample plates or microplates may be read by a plate reader or microplate photometer. According to an embodiment reagent beads, plugs or inserts in a sample well of a sample plate may be illuminated by light having a specific wavelength (optionally selected by an optical filter or a monochromator). As a result of the illumination, captured sample on the reagent bead, plug or insert may absorb light and then either reflect the light which is then detected by a spectrophotometer or emit light (i.e. fluoresce) which may be detected by a light detector. According to a particularly preferred embodiment the plate reader may detect luminescence using a light detector. In particular, reagent beads which have detected an analyte of interest may emit light by a chemiluminescent process. The intensity of light emitted from a reagent bead, plug or insert will slowly decrease with time but the determined intensity can be normalised by also detecting the intensity of light emitted from one or more control reagent beads, plugs or inserts.

A variety of different types of plate reader are known including chromogenic, chemifluorescent and chemiluminescent imaging plate detectors. A chemiluminescent plate reader is particularly preferred.

The sample plate according to the preferred embodiment advantageously reduces crosstalk between neighbouring reagent beads, plugs or inserts when the reagent beads, plugs or inserts are being read by a plate reader by substantially preventing light emitted from one reagent bead, plug or insert being able to impinge upon a neighbouring reagent bead, plug or insert.

Furthermore, the sample plate according to the preferred embodiment is also particularly advantageous compared to known sample plates or multiplexed sample plates comprising spherical reagent beads in that fluid dead zones are prevented from forming when a preferred sample plate is shaken thereby resulting in a more uniform transfer of molecules from a sample fluid to reagent beads, plugs or inserts and wherein the uniform transfer of molecules to the reagent beads, plugs or inserts is irrespective of the reagent bead, plug or insert position.

A yet further advantage of the preferred embodiment is that the preferred reagent beads, plugs or inserts can be positioned so that they are flush with the bottom surface of the sample well thereby enabling a more uniform transfer of molecules from a sample fluid to the reagent beads, plugs or inserts.

According to other embodiments the non-spherical (e.g. generally or substantially cylindrical) reagent beads, plugs or inserts may be inserted so that they are positioned so as to stand slightly proud of (or alternatively recessed below) the bottom surface of the sample well. It is envisaged, for example, that in certain circumstances it may be advantageous for the reagent beads, plugs or inserts to extend or protrude above the bottom surface of the sample well (or alternatively for the upper surface of the reagent beads, plugs or inserts to be recessed below the lower surface of the sample well).

Another advantage of the preferred embodiment is that the preferred reagent beads, plugs or inserts can be produced by an injection moulding process which is more cost effective than the conventional approach of grinding spherical reagent beads. Furthermore, producing preferred reagent beads, plugs or inserts according to the preferred embodiment reduces any effects due to potential contamination of the reagent beads during the manufacturing process.

Another advantage of the preferred embodiment is that preferred non-spherical reagent beads, plugs or inserts can be inserted into holes or apertures provided in the base portion of a sample well using a relatively simple inserter. Advantageously, according to various embodiments it is not necessary to use a relatively complex robotic reagent bead inserter to position reagent beads precisely at a set height within the base portion of the sample well. Instead, according to the preferred embodiment the reagent beads, plugs or inserts can be more simply inserted until the upper surface of the reagent beads, plugs or inserts are flush with the bottom of the sample well.

One or more through holes preferably pass from the bottom of the sample well through to the rear or bottom surface of the sample plate. As a result, if a reagent bead, plug or insert is not retained or secured within the open through hole then any fluid in the sample well can leak out of the sample well via the through hole.

It should be understood that a circular reagent bead, plug or insert within a hole, bore or recess having a square cross-section will not form a fluid-tight circumferential seal with the wall defining the hole, bore or recess. A fluid-tight circumferential seal should be understood as meaning that a barrier is formed around the entire circumference of the bead, plug or insert and the wall defining the hole, bore or recess. According to the preferred embodiment reagent beads, plugs or inserts are retained or secured within a hole, aperture or a recess formed in the base portion of the sample plate. Each reagent bead, plug or insert preferably forms a fluid-tight and/or water-tight and/or air-tight seal about the entire outer diameter or circumference of the reagent bead, plug or insert. It will be understood that the spherical reagent beads in the arrangement disclosed in US 2009/0069200 (Yu) do not form a fluid-tight circumferential seal with the square wall defining the subwell.

Once the reagent bead, plug or insert is located within the hole, aperture or recess, then fluid is substantially prevented from being able to pass from one side of the hole, aperture or recess to the other side by the reagent bead, plug or insert which forms a tight seal about the entire circumference of the reagent bead, plug or insert.

Open through holes or recesses provided in the base portion of a sample well may be substantially cylindrical and may have a diameter less than a diameter of a preferred non-spherical reagent bead, plug or insert deposited in the through hole or the recess so that the non-spherical reagent bead, plug or insert according to a preferred embodiment is retained or secured within the through hole or within the recess by an interference or friction fit.

The open through hole or the recess may according to another embodiment be conical and have a first diameter which is greater than a diameter of a preferred reagent bead, plug or insert deposited in the through hole or in the recess and a second diameter which is less than a diameter of the preferred reagent bead, plug or insert deposited in the through hole or in the recess. As a result, reagent beads, plugs or inserts are secured within the through hole by the taper.

The one or more non-spherical reagent beads, plugs or inserts may be substantially retained or secured, in use, within the one or more holes or apertures so that the upper surface of the one or more reagent beads, plugs or inserts is substantially flush with or co-planar with the upper surface of the base portion.

The one or more reagent beads, plugs or inserts may comprise one or more substantially or generally cylindrical reagent beads, plugs or inserts.

The one or more reagent beads, plugs or inserts may have a substantially or generally circular, round, oval, curved, square, rectangular, polygonal, regular or irregular cross-sectional profile.

The one or more reagent beads, plugs or inserts may comprise one or more substantially prism shaped or prismatic reagent beads, plugs or inserts.

The one or more reagent beads, plugs or inserts may have a cross-sectional profile which either: (i) remains substantially constant along the full longitudinal length of the reagent bead, plug or insert; or (ii) varies, changes or tapers along one or more portions of the longitudinal length of the reagent bead, plug or insert.

The one or more reagent beads, plugs or inserts may have a substantially or generally circular cross-sectional profile wherein the diameter of the one or more reagent beads, plugs or inserts in a middle portion of the reagent beads, plugs or inserts is greater than at one or both end portions of the reagent beads, plugs or inserts.

The one or more reagent beads, plugs or inserts may have a substantially circular cross-sectional profile wherein the diameter of the one or more reagent beads, plugs or inserts tapers or narrows towards one or both end portions of the reagent beads, plugs or inserts.

The one or more reagent beads, plugs or inserts may have a first end face and a second opposed end face, wherein the first end face and/or the second end face are coated with a reagent or include a reagent.

The one or more reagent beads, plugs or inserts are preferably insertable in either a first orientation or a second different orientation into the one or more holes or apertures.

The one or more reagent beads, plugs or inserts are preferably effective irrespective of whether the one or more reagent beads, plugs or inserts are inserted in the first orientation or in the second orientation into the one or more holes or apertures provided in the base portion of the sample plate.

The one or more reagent beads, plugs or inserts may have a first end face, wherein the first end face is coated with a reagent or includes a reagent.

The one or more reagent beads, plugs or inserts are preferably insertable in a first orientation into the one or more holes or apertures.

The one or more reagent beads, plugs or inserts may be effective if the one or more reagent beads, plugs or inserts are inserted in the first orientation into the one or more holes or apertures. According to an embodiment the one or more reagent beads, plugs or inserts may be intended to be inserted in just one orientation into a hole or aperture in the base portion of a sample well.

According to another aspect there is provided a sample plate or multiplexed sample plate comprising one or more sample wells, wherein one or more of the sample wells comprise:

a base portion having an upper surface which forms a bottom portion of the sample well;

one or more holes or apertures provided in the base portion; and

one or more raised portions, flanges, rims or collars surrounding the one or more holes or apertures;

wherein one or more reagent beads, plugs or inserts are substantially retained or secured, in use, within the one or more holes or apertures so as to form a substantially fluid-tight circumferential seal with either a wall of the base portion which defines the hole or aperture and/or the one or more raised portions, flanges, rims or collars.

According to an embodiment if a sample plate is provided having one or more raised portions, flanges, rims or collars surrounding one or more holes or apertures provided in the base portion of the sample plate then one or more spherical reagent beads may be inserted within the one or more holes or apertures.

The one or more holes or apertures may comprise one or more open through holes.

The one or more holes or apertures may be substantially or generally cylindrical.

The one or more holes or apertures may have a substantially or generally circular, round, oval, curved, square, rectangular, polygonal, regular or irregular cross-sectional profile.

The one or more holes or apertures may have a cross-sectional profile which either: (i) remains substantially constant along the full longitudinal length of the hole or aperture; or (ii) varies, changes or tapers along one or more portions of the longitudinal length of the hole or aperture.

The one or more holes or apertures may have a diameter less than a diameter of a reagent bead, plug or insert deposited in the hole or aperture so that the reagent bead, plug or insert is retained or secured within the hole or aperture by an interference or friction fit.

The one or more reagent beads, plugs or inserts may have a circumferential step portion, flange or stopper feature.

The one or more holes or apertures may have a reduced diameter portion and the circumferential step portion, flange or stopper feature of the one or more reagent beads, plugs or inserts may be arranged to abut against the reduced diameter portion so as to position the reagent bead, plug or insert so that the upper surface of the reagent bead, plug or insert does not substantially protrude above or beyond the upper surface of the base portion. Other embodiments are contemplated wherein the circumferential step portion, flange or stopper feature of the one or more reagent beads, plugs or inserts may be arranged to abut against the reduced diameter portion so as to position the reagent bead, plug or insert so that the upper surface of the reagent bead, plug or insert protrudes beyond the upper surface of the base portion or is recessed below the upper surface of the base portion.

The circumferential step portion, flange or stopper feature of the one or more reagent beads, plugs or inserts may abut against the reduced diameter portion in use so as to position the reagent bead, plug or insert so that the upper surface of the reagent bead, plug or insert is substantially flush with or co-planar with the upper surface of the base portion.

The one or more reagent beads, plugs or inserts may have a square upper edge or an edge which in use abuts substantially parallel to or flush with a corresponding surface of the base portion which defines the one or more holes or apertures.

At least a portion or substantially the whole of an upper or first and/or a lower or second face of the one or more reagent beads, plugs or inserts may have a first surface finish or first surface roughness.

At least a portion or substantially the whole of a sealing face, sidewall or surface of the one or more reagent beads, plugs or inserts which contacts a wall which defines the hole or aperture may have a second different surface finish or second different surface roughness.

The second surface finish may be smoother than the first surface finish.

The second surface roughness may be less than the first surface roughness.

The one or more reagent beads, plugs or inserts may be formed by an injection moulding process.

The injection moulding process may leave a seam on at least some of the reagent beads, plugs or inserts.

The reagent beads, plugs or inserts may be inserted in use into the one or more holes or apertures of a sample plate so that the seam on at least some of the reagent beads, plugs or inserts is positioned on, above or below a sealing face, sidewall or surface which contacts a wall which defines the hole or aperture.

The reagent beads, plugs or inserts may be inserted in use into the one or more holes or apertures so that the seam on at least some of the reagent beads, plugs or inserts is part of the portion of the reagent bead, plug or insert which forms a substantially fluid-tight seal circumferential seal with a wall of the base portion which defines the hole or aperture.

The injection moulding process may leave a sprue on at least some of the reagent beads, plugs or inserts.

The reagent beads, plugs or inserts may be inserted in use into the one or more holes or apertures so that the sprue on at least some of the reagent beads, plugs or inserts is positioned on, above or below a sealing face, sidewall or surface which contacts a wall which defines the hole or aperture.

The reagent beads, plugs or inserts may be inserted in use into the one or more holes or apertures so that the sprue on at least some of the reagent beads, plugs or inserts forms part of the portion of the reagent bead, plug or insert which forms a substantially fluid-tight seal circumferential seal with a wall of the base portion which defines the hole or aperture.

The sample plate may comprise an Immunoassay sample plate.

The sample plate may comprise a hybridization probe for detecting the presence of complementary DNA or RNA samples.

According to another aspect there is provided a combination of a sample plate or multiplexed sample plate as described above and one or more non-spherical, spherical or substantially or generally cylindrical reagent beads, plugs or inserts inserted or located in one or more of the holes or apertures of the one or more sample wells.

At least some or substantially all of the reagent beads, plugs or inserts carry, comprise or are otherwise coated with the same or a different reagent, wherein the reagent(s) are arranged and adapted to assay for the same or different analyte(s) of interest in a sample liquid.

At least some or substantially all of the reagent beads, plugs or inserts carry, comprise or are otherwise coated with a nucleic acid probe, wherein the nucleic acid probe is arranged and adapted to hybridize with single-stranded nucleic acid, DNA or RNA.

According to another aspect there is provided a combination of a plate frame holder and a sample plate or multiplexed sample plate as described above.

According to another aspect there is provided an automated apparatus comprising:

one or more reagent bead, plug or insert inserters;

a sample plate or multiplexed sample plate as described above; and

a control system arranged and adapted to control the insertion of reagent beads, plugs or inserts into one or more sample wells of the sample plate or multiplexed sample plate.

According to another aspect there is provided apparatus for assaying a liquid for one or more analytes of interest, the apparatus comprising:

one or more reagent bead, plug or insert inserters; and

a sample plate or multiplexed sample plate as described above.

According to another aspect there is provided a reader for reading an optical or other signal from one or more reagent beads, plugs or inserts which are retained or secured within one or more holes or apertures provided in a base portion of a sample plate or multiplexed sample plate as described above.

According to another aspect there is provided a method comprising:

providing a sample plate or multiplexed sample plate comprising one or more sample wells, wherein one or more of the sample wells comprise a base portion having an upper surface which forms a bottom portion of the sample well with one or more holes or apertures provided in the base portion; and

retaining or securing one or more non-spherical reagent beads, plugs or inserts within the one or more holes or apertures so as to form a substantially fluid-tight circumferential seal with a wall of the base portion which defines the hole or aperture.

An upper surface of the one or more reagent beads, plugs or inserts preferably does not substantially protrude above or beyond the upper surface of the base portion.

According to another aspect there is provided a method comprising:

providing a sample plate or multiplexed sample plate comprising one or more sample wells, wherein one or more of the sample wells comprise a base portion having an upper surface which forms a bottom portion of the sample well, one or more holes or apertures provided in the base portion and one or more raised portions, flanges, rims or collars surrounding the one or more holes or apertures; and

retaining or securing one or more reagent beads, plugs or inserts within the one or more holes or apertures so as to form a substantially fluid-tight circumferential seal with either a wall of the base portion which defines the hole or aperture and/or the one or more raised portions, flanges, rims or collars.

According to another aspect there is provided a method of using a sample plate to analyse a sample for multiple analytes comprising:

providing a sample plate or multiplexed sample plate as described above;

optionally inserting one or more different reagent beads, plugs or inserts into one or more different holes or apertures of a sample well; and

adding a sample to the sample well.

According to another aspect there is provided a method of using an Enzyme Linked ImmunoSorbent Assay (ELISA) to detect an antigen or an antibody in a sample comprising:

providing a sample plate or multiplexed sample plate as described above;

optionally inserting one or more different reagent beads, plugs or inserts into one or more different holes or apertures of a sample well; and

adding a sample to the sample well.

According to another aspect there is provided a method of using a nucleic acid probe to detect a DNA or RNA sequence in a sample comprising:

providing a sample plate or multiplexed sample plate as described above;

optionally inserting one or more different reagent beads, plugs or inserts into one or more different holes or apertures of a sample well; and

adding a sample to the sample well.

According to another aspect there is provided a method for assaying for one or more analytes of interest in a sample comprising:

inserting one or more non-spherical reagent beads, plugs or inserts into one or more holes or apertures of one or more sample wells of a sample plate so as to retain or secure a reagent bead, plug or insert within the hole or aperture so as to form a substantially fluid-tight circumferential seal with a wall of a base portion which defines the hole or aperture.

An upper surface of the one or more reagent beads, plugs or inserts preferably does not substantially protrude above or beyond an upper surface of the base portion.

According to another aspect there is provided a method for assaying for one or more analytes of interest in a sample comprising:

inserting one or more reagent beads, plugs or inserts into one or more holes or apertures of one or more sample wells of a sample plate or multiplexed sample plate having one or more raised portions, flanges, rims or collars surrounding the one or more holes or apertures so as to retain or secure a reagent bead, plug or insert within the hole or aperture so as to form a substantially fluid-tight circumferential seal with either a wall of the base portion which defines the hole or aperture and/or the one or more raised portions, flanges, rims or collars.

According to another aspect there is provided a method of detecting an analyte comprising:

providing a sample plate or multiplexed sample plate as described above wherein one or more reagent beads, plugs or inserts are retained or secured within one or more holes or apertures provided in the base portion of the sample plate;

adding a sample to the sample plate or multiplexed sample plate; and

detecting binding of an analyte in the sample to a reagent bead, plug or insert.

The method preferably further comprises one or more of the following steps:

(i) incubating the sample plate or multiplexed sample plate; and/or

(ii) washing the sample plate or multiplexed sample plate; and/or

(iii) aspirating the sample plate or multiplexed sample plate; and/or

(iv) adding an enzyme conjugate to the sample plate or multiplexed sample plate; and/or

(v) adding a visualising agent to the sample plate or multiplexed sample plate; and/or

(vi) visually analysing the sample plate or multiplexed sample plate; and/or

(vii) reading or determining the intensity of light reflected, transmitted or emitted from individual reagent beads, plugs or inserts in a sample well.

According to another aspect there is provided a kit for performing an Enzyme Linked ImmunoSorbent Assay (ELISA) procedure comprising:

one or more sample plates or multiplexed sample plates as described above; and

a plurality of reagent beads, plugs or inserts wherein the reagent beads, plugs or inserts are coated with or comprise the same or different reagents comprising an antibody, an antigen or another biomolecule.

According to another aspect there is provided a kit for performing a nucleic acid probe procedure comprising:

one or more sample plates or multiplexed sample plates as described above; and

a plurality of reagent beads, plugs or inserts wherein the reagent beads, plugs or inserts are coated with or comprise the same or different DNA or RNA sequence.

One or more reagent beads, plugs or inserts are preferably retained or secured within one or more holes or apertures provided in the base portion of the sample plate.

According to another aspect there is provided a kit for detecting an analyte comprising:

one or more sample plates or multiplexed sample plates as described above; and

a plurality of reagent beads, plugs or inserts retained or secured within one or more through holes or apertures provided in the base portion of the sample plate or multiplexed sample plate so that the plurality of reagent beads, plugs or inserts form a substantially fluid-tight circumferential seal with a wall of the base portion which defines the hole or the aperture.

According to another aspect there is provided a method of manufacturing a sample plate or multiplexed sample plate by injection moulding comprising:

injecting a substrate into a mould to form a sample plate or multiplexed sample plate as described above.

According to another aspect there is provided a method of manufacturing a sample plate or multiplexed sample plate as described above, further comprising inserting one or more same or different reagent beads, plugs or inserts into the one or more holes or apertures so that the one or more reagent beads, plugs or inserts form a substantially fluid-tight circumferential seal with a wall of the base portion which defines the hole or aperture.

According to another aspect there is provided a method of inserting beads, plugs or inserts comprising:

providing a bead, plug or insert inserter;

providing a sample plate or multiplexed sample plate comprising a sample well, wherein the sample well comprises a base portion, wherein the base portion comprises one or more holes or apertures, wherein the one or more holes have a diameter less than a diameter of the bead, plug or insert; and

controlling the insertion of one or more non-spherical reagent beads, plugs or inserts into the sample plate or multiplexed sample plate.

The step of inserting is preferably performed automatically.

According to another aspect there is provided a kit for detecting one or more analytes comprising:

a plurality of non-spherical beads, plugs or inserts; and

a sample plate or multiplexed sample plate comprising a sample well, wherein the sample well comprises a base portion, wherein the base portion comprises one or more holes or apertures, wherein the one or more holes or apertures comprises a diameter less than a diameter of the non-spherical beads, plugs or inserts.

The plurality of reagent beads, plugs or inserts preferably comprise one or more probes.

The probe may be a nucleic acid, antibody, antibody fragment, protein, peptide, aptamer or a chemical compound.

The probe may be an oligonucleotide.

According to another aspect there is provided a method of detecting one or more analytes or biomolecules comprising:

adding a sample to a sample plate or multiplexed sample plate comprising a sample well, wherein the sample well comprises a base portion, wherein the base portion comprises one or more recesses, wherein each recess comprises a probe and each recess has a diameter less than a diameter of a non-spherical reagent bead, plug or insert comprising the probe; and

detecting binding of one or more analytes or biomolecules in the sample with the one or more probes.

The sample plate or multiplexed sample plate may comprise a plurality of probes and a plurality of analytes or biomolecules may be detected.

A plurality of samples may be added to the sample plate.

According to another aspect there is provided a sample plate or multiplexed sample plate comprising one or more sample wells, wherein one or more of the sample wells comprise:

a base portion; and

one or more recesses provided in the base portion;

wherein each of the one or more recesses has a dimension for a non-spherical bead, plug or insert deposited or inserted in the well to be substantially retained or secured within the recess, and the non-spherical bead, plug or insert forms a substantially fluid-tight circumferential seal with a wall of the base portion which defines the recess.

According to another aspect there is provided a kit for detecting an analyte comprising:

a plurality of reagent beads, plugs or inserts; and

sample plate or multiplexed sample plate comprising a sample well, wherein the sample well comprises a base portion, wherein the base portion comprises one or more recesses, wherein each of the one or more recesses has a dimension for a non-spherical bead, plug or insert deposited or inserted in the well to be substantially retained or secured within the recess, and the bead, plug or insert forms a substantially fluid-tight circumferential seal with a wall of the base portion which defines the recess.

According to another aspect there is provided a method of detecting one or more analytes or biomolecules comprising:

adding a sample to a sample plate comprising a sample well, wherein the sample well comprises a base portion, wherein the base portion comprises one or more recesses, wherein each of the one or more recesses has a dimension for a non-spherical bead, plug or insert deposited or inserted in the well to be substantially retained, inserted or secured within the recess, and the bead, plug or insert forms a substantially fluid-tight circumferential seal with a wall of the base portion which defines the recess; and

detecting binding of one or more analytes or biomolecules in the sample with the one or more probes.

According to another aspect there is provided a method of manufacturing comprising:

injecting a resin into a mould so as to form one or more generally or substantially cylindrical reagent beads, plugs or inserts wherein the one or more generally or substantially cylindrical reagent beads, plugs or inserts may be inserted within the one or more holes or apertures of a sample plate as described above.

According to another aspect there is provided a plate reader for determining the intensity or luminosity of one or more non-spherical reagent beads, plugs or inserts retained, inserted or secured within one or more holes or apertures of a sample plate as described above.

According to another aspect there is provided a plate reader for determining the intensity or luminosity of one or more spherical reagent beads, plugs or inserts retained, inserted or secured within one or more holes or apertures of a sample plate as described above.

According to another aspect there is provided a method of inserting one or more reagent beads, plugs or inserts into a sample plate comprising:

providing a sample plate comprising a sample well, wherein the sample well comprises a base portion, wherein the base portion comprises one or more holes or apertures, wherein the one or more holes or apertures have a diameter less than a diameter of a reagent bead, plug or insert; and

partially inserting in a serial or parallel manner one or more non-spherical reagent beads, plugs or inserts into one or more of the holes or apertures; and then

using a press-in tool to simultaneously press in the one or more non-spherical reagent beads, plugs or inserts into the one or more of the holes or apertures.

The method may further comprise using the press-in tool to simultaneously press in the one or more non-spherical reagent beads, plugs or inserts into the one or more of the holes or apertures so that an upper surface of the one or more non-spherical reagent beads, plugs or inserts is substantially flush or co-planar with the bottom surface of the sample well.

According to another aspect there is provided a multiplexed sample plate comprising one or more sample wells, wherein one or more of the sample wells comprise:

a base portion having an upper surface which forms a bottom portion of the sample well; and

a plurality of holes or apertures provided in the base portion; the multiplexed sample plate further comprising:

one or more first non-spherical reagent beads, plugs or inserts substantially retained, inserted or secured within one or more holes or apertures so as to form a substantially fluid-tight circumferential seal with a wall of the base portion which defines the hole or aperture; and

one or more different second non-spherical reagent beads, plugs or inserts substantially retained, inserted or secured within one or more holes or apertures so as to form a substantially fluid-tight circumferential seal with a wall of the base portion which defines the hole or aperture;

wherein an upper surface of the one or more first and second reagent beads, plugs or inserts do not substantially protrude above or beyond the upper surface of the base portion.

Preferably, the one or more first non-spherical reagent beads, plugs or inserts are arranged to test for the presence of a first substance and the one or more second non-spherical reagent beads, plugs or inserts are arranged to test for the presence of a second different substance, analyte or biomolecule.

The multiplexed sample plate may further comprise one or more third non-spherical reagent beads, plugs or inserts substantially retained or secured within one or more holes or apertures so as to form a substantially fluid-tight circumferential seal with a wall of the base portion which defines the hole or aperture, wherein the one or more third non-spherical reagent beads, plugs or inserts are arranged to test for the presence of a third different substance, analyte or biomolecule.

The multiplexed sample plate may further comprise one or more fourth or further non-spherical reagent beads, plugs or inserts substantially retained or secured within one or more holes or apertures so as to form a substantially fluid-tight circumferential seal with a wall of the base portion which defines the hole or aperture, wherein the one or more fourth non-spherical reagent beads, plugs or inserts are arranged to test for the presence of a fourth different substance, analyte or biomolecule.

According to another aspect there is provided a method of manufacturing or assembling a multiplexed sample plate comprising:

inserting one or more first and second non-spherical reagent beads, plugs or inserts into a sample plate comprising a sample well, wherein the sample well comprises a base portion, wherein the base portion comprises one or more holes or apertures, wherein the one or more holes or apertures have a diameter less than a diameter of a reagent bead, plug or insert;

wherein the one or more first non-spherical reagent beads, plugs or inserts are arranged to test for the presence of a first substance, analyte or biomolecule and the one or more second non-spherical reagent beads, plugs or inserts are arranged to test for the presence of a second different substance, analyte or biomolecule.

The through hole or the recess may have a taper selected from the group consisting of: (i) <0.5°; (ii) 0.5°; (iii) 0.5-1°; (iv) 1-2°; (v) 2-4°; (vi) 4-6°; (vii) 6-8°; (viii) 8-10°; and (ix) >10°.

An opening to the through hole or recess is preferably circular.

The through hole or recess may have a circular cross-sectional shape or profile. The through holes or recesses may have a circular cross-section along at least 5%, 10%, 15%, 20%, 25%, 30%, 35%, 40%, 45%, 50%, 55%, 60%, 65%, 70%, 75%, 80%, 85%, 90%, 95% or 100% of the length or depth of the through hole or recess.

The diameter of the through hole may be selected from the group consisting of: (i) <0.5 mm; (ii) 0.5-1.0 mm; (iii) 1.0-1.5 mm; (iv) 1.5-2.0 mm; (v) 2.0-2.5 mm; (vi) 2.5-3.0 mm; (vii) 3.0-3.5 mm; (viii) 3.5-4.0 mm; (ix) 4.0-4.5 mm; (x) 4.5-5.0 mm; (xi) <5.0 mm; and (xii) >5.0 mm.

The depth of the through hole may be selected from the group consisting of: (i) <0.5 mm; (ii) 0.5-1.0 mm; (iii) 1.0-1.5 mm; (iv) 1.5-2.0 mm; (v) 2.0-2.5 mm; (vi) 2.5-3.0 mm; (vii) 3.0-3.5 mm; (viii) 3.5-4.0 mm; (ix) 4.0-4.5 mm; (x) 4.5-5.0 mm; (xi) <5.0 mm; and (xii) >5.0 mm.

According to an embodiment in at least one sample well (or in all the sample wells) the base portion may comprise a plurality of open through holes wherein at least some (or all) of the plurality of open through holes are arranged so that there is no direct line of sight between reagent beads, plugs or inserts retained or secured in adjacent open through holes.

One or more open through holes may comprise a countersunk or enlarged portion for facilitating the insertion of a reagent bead, plug or insert into one or more of the through holes or recesses.

The one or more sample wells preferably comprise at least 1, 2, 3, 4, 5, 6, 7, 8, 9, 10, 11, 12, 13, 14, 15, 16, 17, 18, 19, 20 or 21 through holes which are each arranged and adapted to receive, in use, a reagent bead, plug or insert.

The one or more through holes provided in the base portion may be arranged: (i) circumferentially around a central portion of the sample well; or (ii) with a plurality of through holes or recesses arranged circumferentially around a central through hole or recess; or (iii) in a substantially close-packed manner; or (iv) in a substantially symmetrical or asymmetrical manner; or (v) in a substantially linear or curved manner; or (vi) in a substantially regular or irregular manner; or (vii) in an array; or (viii) in a circle or two or more concentric circles with no through hole or recess located at the centre of the base portion.

The sample plate may comprise sample wells arranged in an A×B format wherein: A is selected from the group consisting of: (i) 1; (ii) 2; (iii) 3; (iv) 4; (v) 5; (vi) 6; (vii) 7; (viii) 8; (ix) 9; (x) 10; and (xi) >10; and B is selected from the group consisting of: (i) 1; (ii) 2; (iii) 3; (iv) 4; (v) 5; (vi) 6; (vii) 7; (viii) 8; (ix) 9; (x) 10; and (xi) >10.

The sample plate may comprise an Immunoassay sample plate.

The sample plate may comprise a hybridization probe for detecting the presence of complementary DNA or RNA samples.

The sample plate may comprise a base having a female, male or other docking portion for securing the sample plate to a corresponding male, female or other docking portion of a plate frame holder.

According to an aspect there is provided a combination of a sample plate or multiplexed sample plate as described above and one or more reagent beads, plugs or inserts inserted or located in one or more of the through holes or recesses of the one or more sample wells.

At least some or substantially all of the reagent beads, plugs or inserts preferably carry, comprise or are otherwise coated with a reagent, wherein the reagent is arranged and adapted to assay for an analyte of interest in a sample liquid.

At least some or substantially all of the reagent beads, plugs or inserts preferably carry, comprise or are otherwise coated with a nucleic acid probe, wherein the nucleic acid probe is arranged and adapted to hybridize with single-stranded nucleic acid, DNA or RNA.

According to another aspect there is provided a combination of a plate frame holder and a sample plate or multiplexed sample plate as described above.

The plate frame holder may comprise a male, female or other docking portion for firmly securing the sample plate to the plate frame holder.

The reagent beads, plugs or inserts may be inserted into one or more of the bores of the sample wells either by the sample plate manufacturer of by the end user.

A bead, plug or insert is substantially retained or secured, in use, within the one or more recesses by an interference or friction fit with the recess or bore or with the circumference of the recess or bore.

A preset force may compress a reagent bead, plug or insert and/or deforms the recess so as to create or enhance an interference or friction fit with the recess or bore.

A reagent bead, plug or insert forms a substantially fluid-tight seal with the recess.

The one or more recesses preferably do not comprise a tapered section.

The sample well may comprise between 2 and 20 recesses.

According to an embodiment the sample well may comprise at least 10 recesses.

The plurality of recesses may be arranged circumferentially around a central portion of the sample well.

According to a less preferred embodiment the central portion may comprise a central recess.

According to the preferred embodiment the central portion does not comprise a recess.

The plurality of recesses are preferably arranged in a substantially symmetrical or regular manner.

According to a less preferred embodiment the plurality of recesses are arranged in a substantially asymmetrical or irregular manner.

According to an embodiment the plurality of recesses are arranged in a substantially linear manner.

According to an embodiment the plurality of recesses are arranged in a substantially curved manner.

The plurality of sample wells are preferably arranged in an A×B format, wherein A and B are perpendicular axes, and the number of wells along the A axis can be greater than, less than, or equal to the number of wells along the B axis.

According to an embodiment the number of wells along the A axis or B axis is at least 2.

The number of wells along the A axis or B axis is preferably between 2 and 15.

According to an embodiment at least one of the plurality of sample wells is connected to another sample well of plurality of samples wells by a frangible region.

The sample plate may comprise a base comprising a docking portion for securing the sample plate to a corresponding docking portion of a plate frame holder.

According to an embodiment the sample plate further comprises a bead.

The bead is preferably attached to a probe.

The probe is preferably a nucleic acid, antibody, antibody fragment, protein, peptide, aptamer or a chemical compound. According to an embodiment the probe is an oligonucleotide.

The bore having the tapered section should not be misconstrued as being, for example, a shallow or small depression in which a reagent bead or microsphere simply can rest but in which the reagent bead or microsphere is not substantially retained or secured.

The sample plate according to the present invention is particularly advantageous compared to the sample plate disclosed in U.S. Pat. No. 5,620,853 (Chiron Corporation).

According to various embodiments, in use, a reagent bead, plug or insert is substantially retained or secured within the bore by an interference or friction fit with the tapered section of the bore.

Reagent beads, plugs or inserts may be inserted into a sample plate having a plurality of tapered holes or sections which act to firmly secure or lock the reagent beads in position once inserted. A preset force may be used to insert the reagent beads, plugs or inserts. The preset force may be sufficient to compress the reagent bead, plug or insert and/or to deform the tapered section of the bore so as to create or enhance the interference or friction fit with the tapered section of the bore.

The sample plate or multiplexed sample plate is particularly robust during manufacture and in subsequent processing stages including the stage of inserting reagent beads, plugs or inserts into the tapered holes and subsequent handling and processing of the sample plate or multiplexed sample plate. Once the reagent beads, plugs or inserts have been inserted into a sample plate or multiplexed sample plate then they are preferably not free to move in any direction and essentially become a fixed part of the sample plate or multiplexed sample plate.

The angle of the taper may be arranged so that reagent beads are locked or are otherwise firmly secured into the holes making the arrangement very reliable.

A reagent bead, plug or insert may be substantially retained or secured within the bore if the sample plate (i.e. the plane of the sample plate) is tipped by more than 10°, 20°, 30°, 40°, 50°, 60°, 70°, 80°, or 90° to horizontal, or is inverted.

The opening to the bore and/or cross-sectional shape of the bore (i.e. at a location intermediate the opening to the bore and the base of the bore) may be circular. However, according to other embodiments the opening and/or cross-sectional shape of the bore may be substantially circular, elliptical, oblong, triangular, square, rectangular, pentagonal, hexagonal, septagonal, octagonal, nonagonal, decagonal or polygonal.

The diameter of the opening of the bore may be selected from the group consisting of: (i) <0.5 mm; (ii) 0.5-1.0 mm; (iii) 1.0-1.5 mm; (iv) 1.5-2.0 mm; (v) 2.0-2.5 mm; (vi) 2.5-3.0 mm; (vii) 3.0-3.5 mm; (viii) 3.5-4.0 mm; (ix) 4.0-4.5 mm; (x) 4.5-5.0 mm; (xi) <5.0 mm; and (xii) >5.0 mm.

According to the preferred embodiment a diameter of the bore, preferably at a location intermediate the opening of the bore and the base of the bore, is preferably at least 5% smaller than the diameter of the reagent bead, plug or insert and/or is preferably at least 5% smaller than the diameter of the opening of the bore. If the bore has a cross-sectional shape that is other than circular, then the smallest span of the cross-sectional shape of the bore, preferably at a location intermediate the opening of the bore and the base of the bore, is preferably at least 5% smaller than the diameter of the reagent bead or microsphere and/or is preferably at least 5% smaller than the diameter of the opening of the bore.

According to various embodiments a diameter of the bore, preferably at a location intermediate the opening of the bore and the base of the bore, is preferably selected from the group consisting of: (i) <0.5 mm; (ii) 0.5-1.0 mm; (iii) 1.0-1.5 mm; (iv) 1.5-2.0 mm; (v) 2.0-2.5 mm; (vi) 2.5-3.0 mm; (vii) 3.0-3.5 mm; (viii) 3.5-4.0 mm; (ix) 4.0-4.5 mm; (x) 4.5-5.0 mm; (xi) <5.0 mm; and (xii) >5.0 mm.

The tapered section of the bore may be substantially linearly tapered. For example, the diameter or circumference of the bore preferably varies (e.g. decreases) substantially linearly with the depth of the bore. If the bore has a cross-sectional shape that is other than circular, then a cross-sectional dimension (e.g. the smallest span of the cross-sectional shape of the bore) or the perimeter of the cross-sectional shape of the bore preferably varies (e.g. decreases) substantially linearly with the depth of the bore.

The reagent beads, plugs or inserts are preferably opaque and signal is preferably only taken from the top of the bead, plug or insert. The bottom of the bead, plug or insert below a press fit or interference fit line preferably does not come into contact with sample fluid. In the preferred embodiment, in use, a reagent bead, plug or insert preferably forms a substantially fluid-tight seal with either the cylindrical or tapered section of the bore, preferably so as to substantially prevent fluid from flowing from the sample well past the reagent bead. A sample plate with inserted reagent beads, plugs or inserts according to various embodiments therefore resembles an empty conventional sample well.

The reagent beads, plugs or inserts preferably do not protrude above the bottom of the sample well thereby avoiding forming a moat region around the upper portion of the bead which could trap fluid.

The reagent beads, plugs or inserts may be arranged so as not to protrude above the bottom of the sample well in which case they are also preferably protected and are not susceptible to damage through handling, pipetting or washing.

Beads, plugs or inserts are pressed or inserted into the pockets, recesses or bores formed in the base portion of the sample wells. The tops of the reagent beads, plugs or inserts once inserted are preferably flush or level with the bottom of the sample well.

The depth of the bore may be selected from the group consisting of: (i) <0.5 mm; (ii) 0.5-1.0 mm; (iii) 1.0-1.5 mm; (iv) 1.5-2.0 mm; (v) 2.0-2.5 mm; (vi) 2.5-3.0 mm; (vii) 3.0-3.5 mm; (viii) 3.5-4.0 mm; (ix) 4.0-4.5 mm; (x) 4.5-5.0 mm; (xi) <5.0 mm; and (xii) >5.0 mm.

An advantageous aspect of the disclosed embodiments is that since the reagent beads, plugs or inserts may be arranged to be inserted so that they are flush with the bottom of the well then the sample plate or multiplexed sample plate can be used with known automated microplate processing systems requiring only minimal hardware modifications. Furthermore, the sample well or multiplexed sample plate according to such an embodiment is essentially a cylinder having proportions which are similar to that of a well of a conventional microplate so the fluid and other handling characteristics of the sample well are well known. Processing steps according to such an embodiment such as pipetting, mixing, washing and incubation preferably follow the same type of fluid characteristics that conventional microplates go through.

The sample plate or multiplexed sample plate according to the preferred embodiment preferably has a fluid capacity of approximately 800 μl but advantageously, in use, only a small fraction of the total fluid capacity of a sample well is required in order to cover all the reagent beads, plugs or inserts disposed in the base of the sample plate.

Another advantageous feature of the sample plate or multiplexed sample plate according to the preferred embodiment is that fluid can be dispensed directly into the centre or central region of a sample well and according to the preferred embodiment the sample plate may be arranged so that no pockets, recesses or bores for securing reagent beads are arranged in the central region of the sample well. Such an arrangement is particularly advantageous in that reagent which preferably coats the reagent beads, plugs or inserts is not inadvertently washed off the reagent beads by the force of the fluid jet from a wash head or pipette tip.

The sample plate or multiplexed sample plate according to various embodiments preferably enables multiple tests to be carried out in a single sample well. This is achieved by inserting different reagent beads, plugs or inserts into separate bores in the same sample well thereby enabling multiplexing to be performed. Reagent beads, plugs or inserts can be pressed into tapered or non-tapered holes in the base of the well as desired which results in a high degree of flexibility and the ability to use the entire sample well with a high efficiency.

A sample plate or multiplexed sample plate according to various embodiments may comprise one or more 12 mm diameter sample wells. Each sample well may have a cross sectional surface area of 58 mm² and in total 54 sample wells of this size can be fitted into a conventional microplate footprint. Within each sample well a varied number of beads, plugs or inserts can be inserted. The bores in a sample well can have different diameters to accommodate different size reagent beads, plugs or inserts if desired.

According to other embodiments one or more sample wells may comprise 6×3.0 mm diameter pockets, recesses or bores, 10×2.0 mm diameter pockets, recesses or bores or 21×1.75 mm pockets, recesses or bores. The central region of the sample well is preferably kept free of pockets, recesses or bores. The pockets, recesses or bores may be arranged in a circle or two or more concentric circles or other patterns about the central region of the sample well.

A sample plate or multiplexed sample plate having an array of 9×6 sample wells may be provided. If six pockets, recesses or bores are provided per sample well, then the sample plate can accommodate 324 reagent beads per plate. If 10 pockets, recesses or bores are provided per sample well, then the sample plate can accommodate 540 reagent beads per plate. If 21 pockets, recesses or bores are provided per sample well, then the sample plate can accommodate 1134 reagent beads per plate.

A further advantageous aspect of the present invention is that the sample plate or multiplexed sample plate according to the present invention is relatively simple to manufacture compared with other known arrangements. The sample plate or multiplexed sample plate can be manufactured by moulding using an open and shut tool so that the manufacturability is high and reliable. The injection mould tool design used to form the sample plates or multiplexed sample plates is simple and does not require the use of undercuts or thin features to mould. As a result, the production of sample plates or multiplexed sample plates having different formats can be readily achieved. A tool that produces a sample well with six pockets or bores can be readily adapted to produce a sample well having a different number (e.g. 21) of pockets.

Another advantage of the preferred embodiment is that validation of different well designs and formats can be achieved simply since the test protocols can remain essentially the same. Pipetting and incubation do not change and the washing procedure only requires, at most, a minor alteration to the aspirate routine.

It is apparent, therefore, that the sample plate or multiplexed sample plate according to the present invention is particularly advantageous compared to other known sample plates such as the sample plate disclosed in U.S. Pat. No. 5,620,853 (Chiron Corporation).

The tapered section or bore may have a taper selected from the group consisting of: (i) <0.5°; (ii) 0.5°; (iii) 0.5-1°; (iv) 1-2°; (v) 2-4°; (vi) 4-6°; (vii) 6-8°; (viii) 8-10°; and (ix) >10°. Alternatively, through holes or bores provided in the base portion may be cylindrical and non-tapered.

The pockets or recesses provided in the base portion may comprise a chamber having a retention member, membrane, lip or annular portion. A reagent bead, plug or insert may be inserted, in use, past or through the retention member, membrane, lip or annular portion into the chamber and may be substantially retained or secured within the chamber by the retention member, membrane, lip or annular portion.

The one or more pockets, recesses or bores may comprise a countersunk or enlarged portion for facilitating the insertion of a reagent bead or microsphere into one or more of the pockets, recesses or bores.

The one or more sample wells preferably comprise at least 1, 2, 3, 4, 5, 6, 7, 8, 9, 10, 11, 12, 13, 14, 15, 16, 17, 18, 19, 20 or 21 pockets or recesses each comprising a bore having a tapered or non-tapered section and which are each arranged and adapted to receive, in use, a reagent bead, plug or insert.

The one or more pockets, recesses or bores provided in the base portion are preferably arranged: (i) circumferentially around a central portion of the sample well; and/or (ii) with a plurality of pockets or recesses arranged circumferentially around one more central pockets or recesses; and/or (iii) in a substantially close-packed manner; and/or (iv) in a substantially symmetrical or asymmetrical manner; and/or (v) in a substantially linear or curved manner; and/or (vi) in a substantially regular or irregular manner; and/or (vii) in an array; and/or (viii) in a circle or two or more concentric circles with no pocket, recess or bore located at the centre of the base portion.

The sample plate is preferably fabricated or otherwise made from polystyrene. The sample plate may comprise either a strip or an array format. For example, according to a preferred embodiment the sample plate may comprise a 6×1 strip of sample wells. According to another preferred embodiment the sample plate may comprise nine 6×1 sample strips of sample wells.

According to an embodiment one or more of the sample wells may be interconnected to one or more other sample wells by one or more frangible regions or connections so that the sample plate can be separated by a user into a plurality of smaller sample plates, sample strips or individual sample wells. This enables a sample plate to be snapped or broken into a plurality of smaller sample plates. For example, a 6×1 strip of sample wells may be snapped into six individual sample wells or into two 3×1 sample strips.

According to an embodiment individual sample wells, sample strips and sample plates may be made from polypropylene. Sample wells, sample strips and sample plates are preferably made from a non-binding material such as polypropylene to ensure non-specific binding in the well is kept to a minimum.

A plate frame may be provided which is arranged to hold a plurality of sample wells, sample strips or one or more sample plates or multiplexed sample plates. The plate frame may be from a plastic such as Acrylonitrile Butadiene Styrene (“ABS”). The plate frame is preferably made from a material which provides high rigidity and which ensures that sample wells, sample strips or one or more sample plates are held securely in place and remain flat after sample wells, sample strips or sample plates are secured into the plate frame. The plate frame is sufficiently robust to withstand handling by a user.

One or more of the sample wells may be interconnected to one or more other sample wells by one or more frangible regions or connections so that the sample plate can be separated by a user into a plurality of smaller sample plates, sample strips or individual sample wells.

According to an aspect there is provided a computer program executable by the control system of an automated apparatus, the automated apparatus comprising one or more reagent bead, plug or insert inserters, wherein the computer program is arranged to cause the control system:

(i) to control the insertion of reagent beads, plugs or inserts into one or more sample wells of a sample plate or multiplexed sample plate as disclosed above.

According to an aspect there is provided a computer readable medium comprising computer executable instructions stored on the computer readable medium, the instructions being arranged to be executable by a control system of an automated apparatus, the automated apparatus comprising one or more reagent bead, plug or insert inserters, wherein the computer program is arranged to cause the control system:

(i) to control the insertion of reagent beads, plugs or inserts into one or more sample wells of a sample plate or multiplexed sample plate as disclosed above.

The computer readable medium is preferably selected from the group consisting of: (i) a ROM; (ii) an EAROM; (iii) an EPROM; (iv) an EEPROM; (v) a flash memory; (vi) an optical disk; (vii) a RAM; and (viii) a hard disk drive.

At least some or substantially all of the reagent beads, plugs or inserts which are inserted, in use, into one or more of the pockets, recesses or bores carry or comprise a reagent, wherein the reagent is arranged and adapted: (i) to analyse samples; and/or (ii) to analyse samples by nucleic acid amplification reactions; and/or (iii) to analyse samples by polymerase chain reactions (PCR); and/or (iv) to analyse samples by an immunoassay process; and/or (v) to analyse samples by using a hybridization probe technique. According to a preferred embodiment different reagent beads, plugs or inserts are inserted into the sample plate or multiplexed sample plate so that a sample deposited into a sample well can be subjected to tests for multiple different analytes, substances or biomolecules of interest.

At least some or substantially all of the reagent beads or microspheres which are inserted, in use, into one or more of the pockets, recesses or bores comprise polystyrene, plastic or a polymer.

The sample plate or multiplexed sample plate disclosed herein preferably comprises multiple beads, plugs or inserts which may be coated with different reagents. The bead, plug or insert composition is dependent on the type of assay being performed. The beads, plugs or inserts may be composed of plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles, Teflon® or any combination thereof. In one embodiment, a bead, plug or insert may comprise polystyrene, plastic, a polymer or a combination thereof. In another embodiment, the bead, plug or insert may comprise a ferrous or magnetic coating or may have a ferrous or magnetic property. Alternatively, the bead, plug or insert may comprise an anti-static coating or has an anti-static property. The beads, plugs or inserts may be translucent, slightly translucent or opaque.

The beads, plugs or inserts may be of irregular shape. In addition, the beads, plugs or inserts may be porous. The bead, plug or insert size may range from nanometers to millimeters. The bead, plug or insert may have a diameter of at least 0.1 mm. The bead, plug or insert may have a diameter of between 0.1 mm and 10 mm. In one embodiment, the bead, plug or insert may have a diameter of greater than about 0.5 mm; 0.5-1.0 mm; 1.0-1.5 mm; 1.5-2.0 mm; 2.0-2.5 mm; 2.5-3.0 mm; 3.0-3.5 mm; 3.5-4.0 mm; 4.0-4.5 mm; 4.5-5.0 mm; or greater than about 5.0 mm. The bead, plug or insert may have a diameter greater than, equal to, or less than the diameter of a recess, pocket or bore of a sample well. For example, the bead, plug or insert may have a diameter less than the diameter of a recess, pocket, or bore of a sample well, wherein the recess, pocket or bore comprises a tapered section. In yet another embodiment, the bead, plug or insert may have a diameter greater than the diameter of a recess, pocket or bore of a sample well. For example, the recess, pocket or bore may not comprise a tapered section. The diameter of a bead, plug or insert to be deposited, or present, in the sample plate, can be at least about 5, 10, 15, 20, 35, 30, 35, 40, 45, 50, 55, 60, 65, 70, 75, 80, 85, 90, 95, or 100% greater than the diameter of a recess of the sample plate. In one embodiment, the bead, plug or insert present in a sample plate does not touch the bottom of a sample plate, such as a base portion of a sample well.

The beads, plugs or inserts within the sample plate or multiplexed sample plate may comprise a reagent or probe, or may be coated with a reagent or probe. The reagent or probe can be used to analyze a sample, such as by detecting one or more analytes, biomolecules or substances. The probe or reagent may be attached to the bead, plug or insert. The attachment can be by a covalent or non-covalent interaction. The probe may comprise a nucleic acid, antibody, antibody fragment, protein, peptide, aptamer or a chemical compound. For example, the probe can be an oligonucleotide. In one embodiment, the probe can be used to detect one or more analytes, biomolecules or substances in a biological sample. In yet another embodiment, the probe can be used to for drug screening. For example, a library of compounds or antibodies can be screened for its binding ability to a protein or nucleic acid probe. According to various embodiments a multiplexed sample plate is provided comprising multiple different reagent beads, plugs or inserts which are arranged to test for the presence of different analytes, biomolecules or substances.

The probe can be used to provide detect a biomarker for a diagnosis or prognosis of a disease or condition, drug response or potential drug response or for monitoring the progression of a disease or condition. For example, the probe can be an antibody or fragment thereof that is used to detect an antigen that is a biomarker for cancer. In another embodiment, the probe can be an antigen, peptide or protein, which is used to detect an antibody in a sample, which can be an indicative of a disease or condition. Accordingly, a multiplexed sample plate may be provided which can test for different biomarkers or biomolecules.

The sample plate or multiplexed sample plate disclosed herein may comprise a plurality of probes, wherein a subset of the plurality of probes differs from another subset of the plurality of probes. The plurality of probes may be attached to beads, plugs or inserts. The different probes may be used to detect different analytes, thus allowing multiplexing with the sample plates or multiplexed sample plates disclosed herein. The sample plate or multiplexed sample plate may comprise at least 2, 3, 4, 5, 6, 7, 8, 9, 10, 11, 12, 13, 14, 15, 16, 17, 18, 19, or 20 different probes. The probes may be of the same type (for example, different antibodies) or of a different type (for example, a combination of nucleic acid probe(s) and antigen(s)).

The apparatus preferably further comprises a translation stage for moving the sample plate or multiplexed sample plate relative to one or more reagent bead, plug or insert inserters or other devices.

The control system is preferably arranged and adapted to control the translation stage so that one or more reagent beads, plugs or inserts from a reagent bead, microsphere, plug or insert inserter are inserted sequentially into different holes or apertures in the sample plate or multiplexed sample plate by moving the sample plate relative to the inserter.

According to an embodiment the apparatus may further comprise a fluid dispensing device for dispensing fluid into the sample wells of a sample plate or multiplexed sample plate.

The fluid dispensing device may be arranged and adapted to dispense x ml of fluid at a time into one or more fluid receiving areas of one or more sample wells, wherein x is preferably selected from the group consisting of: (i) <10; (ii) 10-20; (iii) 20-30; (iv) 30-40; (v) 40-50; (vi) 50-60; (vii) 60-70; (viii) 70-80; (ix) 80-90; (x) 90-100; (xi) 100-110; (xii) 110-120; (xiii) 120-130; (xiv) 130-140; (xv) 140-150; (xvi) 150-160; (xvii) 160-170; (xviii) 170-180; (xix) 180-190; (xx) 190-200; and (xxi) >200.

The apparatus preferably further comprises an image analysis device or camera for determining whether or not a reagent bead, plug or insert has been inserted into a pocket, recess or bore of the sample plate.

The sample plate may have a first colour (or may be transparent) and the reagent beads, plugs or inserts may have a second different colour which preferably contrasts with the first colour (or transparency) in order to facilitate visual detection of the presence or absence of a reagent bead, plug or insert in a pocket, recess or bore of the sample plate.

According to an embodiment the sample plate may further comprise a luminescence or fluorescence marker.

The apparatus may further comprise a luminescence or fluorescence detecting device for determining whether or not a reagent bead, plug or insert has been inserted into a pocket, recess or bore of the sample plate by determining whether or not a reagent bead, plug or insert obstructs or partially obstructs the luminescence or fluorescence marker.

The apparatus may further comprise a magnetic and/or electrical and/or capacitive and/or mechanical sensor for sensing whether or not a reagent bead, plug or insert has been dispensed or is otherwise present in a pocket, recess or bore of a sample plate.

The control system may determine the number of reagent beads, plugs or insert present and/or the number of reagent beads, plugs or inserts absent and/or the number of reagent beads, plugs or inserts inserted and/or the number of reagent beads, plugs or inserts desired to be (or remaining to be) inserted into a sample well.

The control system may measure and/or adjust the volume of fluid dispensed or desired to be dispensed into a sample well dependent upon the number of reagent beads, plugs or inserts determined to be present and/or absent and/or inserted and/or desired to be inserted into a sample well.

The control system may be arranged and adapted to ensure that the upper surface of at least some or substantially all reagent beads, plugs or inserts located in the bores of a sample well are at least partially or fully immersed by a fluid when a fluid is dispensed into the sample well.

The control system may be arranged and adapted to ensure that the height of fluid dispensed into a sample well remains substantially constant irrespective of the number of reagent beads, plugs or inserts present, absent, inserted or desired to be inserted into a sample well.

### BRIEF DESCRIPTION OF THE DRAWINGS

Various embodiments of the present invention together with other arrangements given for illustrative purposes only will now be described, by way of example only, and with reference to the accompanying drawings in which:

FIG. 1 shows a sample well of a known sample plate;

FIG. 2A shows a plan view of a sample well of a known sample plate, FIG. 2B shows in greater detail the bottom of a known sample well and FIG. 2C shows a reagent bead or microsphere dispensed in a pocket of a known sample well;

FIG. 3 shows a known microarrayer or automated apparatus;

FIG. 4A shows a known arrangement comprising nine sample strips loaded into a plate frame, wherein each sample strip comprises a 6×1 array of sample wells and FIG. 4B shows a known plate frame into which a sample plate or one or more sample strips may be loaded;

FIG. 5A shows in greater detail a known sample strip comprising six sample wells and FIG. 5B shows a known sample strip comprising six sample wells being loaded into a plate frame;

FIG. 6A shows a single well being loaded into a plate frame, FIG. 6B shows in greater detail two sample wells connected by a break apart feature, FIG. 6C shows a sample well having an end feature and FIG. 6D shows a sample well having an ID and orientation tab;

FIG. 7A shows the underneath of a strip of sample wells, FIG. 7B shows a female alignment and retaining feature which helps to align a sample strip or sample well with a plate frame and FIG. 7C shows a corresponding male alignment and retaining feature which is provided in the base of the plate frame;

FIG. 8 shows a cross-sectional view of a known strip of sample wells and shows an arrangement wherein the sample wells have a plurality of tapered bores wherein the angle of the taper is 6.0°;

FIG. 9A shows a known arrangement wherein conical through holes are provided in the base portion of a sample plate and reagent beads are loaded from the rear of the sample plate and FIG. 9B shows a sample plate wherein the sample plate has a cylindrical non-tapered through hole such that reagent beads may be loaded or inserted from the top through the sample well and are secured within the through hole by an interference fit;

FIG. 10 shows a known sample strip comprising six sample wells wherein reagent beads are fitted from the underneath of the sample plate;

FIG. 11 shows a cross sectional 3D view of a known arrangement showing reagent beads located within a concave end portion of a through hole;

FIG. 12 shows a known cartridge holding assembly;

FIG. 13 shows a section through the known cartridge holding assembly;

FIG. 14 shows a known reagent bead cartridge;

FIG. 15 shows the inside of a known reagent bead cartridge;

FIG. 16 shows in greater detail silicone membranes in the base of a known reagent bead cartridge;

FIG. 17 shows push rods and a cartridge holder assembly;

FIG. 18 shows connection bosses at a lower end of push rods in greater detail;

FIG. 19 shows the upper ends of the push rods in greater detail;

FIG. 20 shows the end of a push rod in greater detail;

FIG. 21 shows a known cartridge holding assembly in a lift mechanism;

FIG. 22 shows in more detail how a lift mechanism may move into engagement with connection bosses of push rods;

FIG. 23 shows in more detail push rods clamped to the lift mechanism;

FIG. 24 illustrates the problem of crosstalk between neighbouring spherical reagent beads according to a conventional arrangement;

FIG. 25 shows cylindrical reagent beads, plugs or inserts according to a preferred embodiment inserted within a bore or through hole of a sample plate and wherein light reflected from a cylindrical reagent bead, plug or insert does not impact or impinge upon a neighbouring reagent bead, plug or insert;

FIG. 26 shows comparative data illustrating how spherical reagent beads located in bores or through holes of a conventional sample plate may pick up approximately 0.44% stray light and illustrate that a significant improvement is obtained according to a preferred embodiment by using cylindrical reagent beads, plugs or inserts which are inserted so as to be flush with the base portion of sample well, wherein a cylindrical reagent bead, plug or insert only picks up 0.04% of stray light;

FIG. 27 shows a cylindrical bead according to a preferred embodiment;

FIG. 28 shows a cylindrical bead according to a preferred embodiment inserted within a bore of a sample well;

FIG. 29 shows a stepped bead, plug or insert inserted within a bore of a sample well according to an embodiment;

FIG. 30 shows a conventional arrangement wherein a spherical bead extends a distance or height of 0.6858 mm above the base portion of a sample well;

FIG. 31 shows an embodiment wherein the base portion of a sample well comprises an additional flange which serves the purpose of reducing the bead height or the exposed height of a reagent bead;

FIG. 32A shows a sample well cut away for illustrative purposes and wherein cylindrical beads, plugs or inserts are partially initially inserted and FIG. 32B shows a sample well cut away for illustrative purposes wherein the cylindrical beads, plugs or inserts are fully inserted using a press-in tool;

FIG. 33 shows a sample well cut away for illustrative purposes with a cylindrical bead, plug or insert inserted according to a preferred embodiment so as to be flush or co-planar with the base portion of a sample well;

FIG. 34 shows spherical beads protruding into the bottom of a sample well;

FIG. 35 shows spherical beads being agitated; and

FIG. 36 shows dead zones around conventional spherical reagent beads after being agitated.

### CONVENTIONAL SAMPLE PLATE

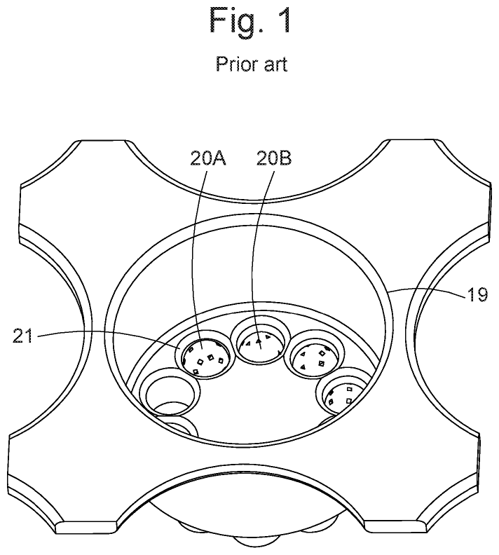

A known arrangement will first be described with reference to FIG. 1. FIG. 1 shows a conventional sample plate which comprises a plurality of sample wells 19. The sample plate may comprise, for example, a 9×6 array of sample wells 19. A single sample well 19 is shown in FIG. 1 for ease of illustration. The sample plate may comprise a strip of sample wells 19 e.g. the sample plate may comprise, for example, a sample strip comprising an 1×9 or an 1×6 array of sample wells 19.

Each sample well 19 comprises a plurality of pockets, recesses or bores 21 which are provided in the base of the sample well 19. In the particular arrangement shown in FIG. 1 the sample well 19 comprises ten pockets, recesses or bores 21 which are formed or otherwise provided in the base of a sample well 19.

The pockets, recesses or bores 21 may be provided around the edge or perimeter of the sample well 19 and the centre or central region of the base of the sample well 19 may be substantially flat and free from pockets, recesses or bores 21.

A plurality of reagent beads or microspheres each having a diameter of 1.75 or 2 mm may be loaded into a reagent bead or microsphere dispenser. A reagent bead or microsphere dispenser may be provided which is arranged to handle reagent beads or microspheres having a diameter other than 1.75 mm or 2 mm. Arrangements are also contemplated wherein reagent beads or microspheres loaded into a particular reagent bead or microsphere dispenser may comprise a plurality or mixture of different diameters.

The reagent beads or microspheres may be pre-loaded or pre-inserted into the pockets, recesses or bores 21 by a sample plate manufacturer. Alternatively, an end-user may load or insert the reagent beads or microspheres into the pockets, recesses or bores 21.

The reagent beads or microspheres may comprise a polystyrene, plastic or polymer core. The reagent beads or microspheres may be coated with a reagent (e.g. an antibody or antigen) which is preferably used to analyse samples. The reagent may be used to analyse samples by polymerase chain reactions (“PCR”) or as part of an immunoassay procedure. Alternatively, the reagent may comprise a DNA or RNA sequence which is used as a hybridization probe to detect the presence of complementary DNA or RNA sequences in a sample. The reagent beads or microspheres may also be coated with an anti-static coating or may have an anti-static property. Different reagent beads or microspheres may be inserted into different bores 21 of a sample well 19 in order to test for different analytes, biomolecules or substances. Accordingly, a multiplexed sample plate may be provided.

A fluid or sample to be tested may be dispensed into a sample well 19 of a sample plate. The fluid may, for example, comprise a sample of blood, serum, saliva or urine taken from a patient.

According to an arrangement 10-200 ml of fluid sample may be dispensed into each sample well 19 of a sample plate.

A control system may be used to determine the location and/or type of reagent beads or microspheres which have been dispensed or inserted into the bores 21 of a sample well 19. Alternatively, the reagent beads or microspheres may have been pre-loaded into the bores 21 of the sample wells 19 by the manufacturer. The control system may also determine into which bores 21 (if any) additional reagent beads or microspheres need to be dispensed or inserted. Once sample fluid has been dispensed into a sample well 19, the control system may check that an appropriate amount of sample fluid has been dispensed and that all the reagent beads or microspheres are at least partially or are fully immersed by the sample fluid.

The volume of sample fluid to be dispensed into a sample well 19 may depend upon the number of bores 21 formed within a sample well 19, the diameter of the reagent beads or microspheres which are dispensed, inserted or pre-loaded into the bores 21 and the extent to which reagent beads or microspheres protrude into the bottom of the sample well 19. The control system may be used to vary the amount of sample fluid dispensed into a sample well 19 so that reagent beads or microspheres are immersed in sample fluid to a substantially constant depth irrespective of the number of bores present in a sample well 19, the diameter of the reagent beads or microspheres or the extent to which the reagent beads or microspheres protrude into the base section of the sample well 19.

Different formats of sample plates may be used. For example, a sample plate may comprise a two dimensional array of sample wells 19 e.g. the sample plate may comprise a 4×4, 4×6, 4×8, 4×10, 4×12, 6×6, 6×8, 6×10, 6×12, 8×8, 8×10, 8×12, 10×10, 10×12 or 12×12 array of sample wells 19. Alternatively, the sample plate may comprise a single dimensional strip of sample wells 19 e.g. the sample plate may comprise a 4×1, 6×1, 8×1, 10×1 or 12×1 strip of sample wells 19.

At least some or all of the pockets, recesses or bores 21 which are provided in the base of a sample well 19 may comprise a bore which may be tapered along at least a portion or substantially the whole of its length. The pockets, recesses or bores 21 may, for example, be arranged to have a 6° taper. The top (or reagent bead or microsphere receiving portion) of a tapered bore may have a diameter of 1.82 mm. The base of the sample well 19 surrounding the bore may be arranged to have a countersunk portion in order to facilitate the insertion of a reagent bead or microsphere into the pocket, recess or bore 21. According to an embodiment the outer diameter of the countersunk portion may be 2.25 mm.

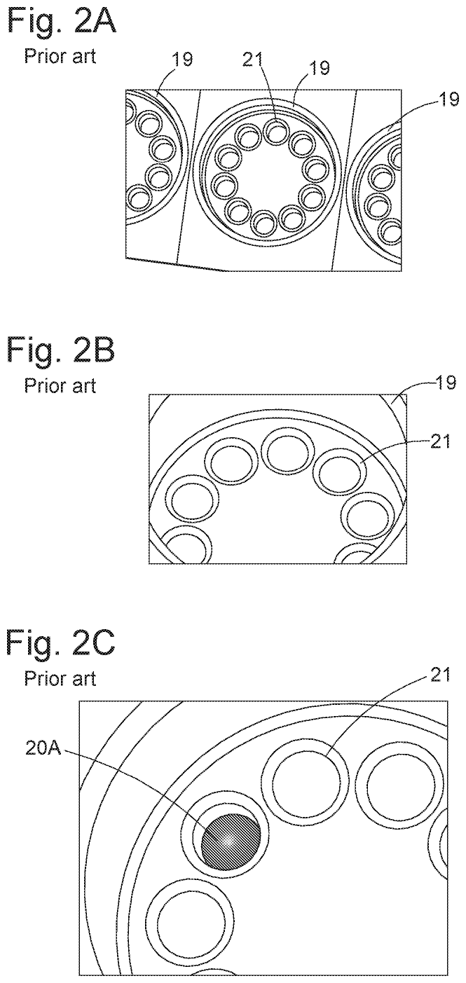

FIG. 2A shows a plan view of a sample well 19 and portions of two adjacent sample wells 19 which are provided in a sample plate. The sample wells shown in FIG. 2A form part of an array of sample wells 19 which are provided in the sample plate. Each of the sample wells 19 shown in FIG. 2A comprise ten pockets, recesses or bores 21 which are disposed in the bottom or base portion of the sample well 19. In use reagent beads or microspheres are preferably inserted into each of the pockets, recesses or bores 21 of a sample well 19 and with the embodiment shown in FIGS. 2A-2C the reagent beads or microspheres are preferably secured in the pockets, recesses or bores 21 by virtue of the diameter of the bore tapering and becoming restricted.

FIG. 2B shows in greater detail the bottom of a sample well 19 and shows a plurality of pockets, recesses or bores 21 provided in the bottom portion of the sample well 19 each of which are arranged and adapted to receive a reagent bead or microsphere. Each of the pockets, recesses or bores 21 provided in the base of the sample well 19 preferably also comprises a countersunk portion or region at the entrance to each tapered bore.

A single reagent bead or microsphere is dispensed and inserted into each pocket, recess or bore 21.

FIG. 2C shows in further detail a reagent bead or microsphere 20A disposed and securely located in a pocket, recess or bore 21 provided in the base of a sample well 19. The reagent bead or microsphere 20A is secured within the pocket, recess or bore 21. According to the embodiment shown in FIG. 2C the upper surface of the reagent bead or microsphere 20A when secured, inserted or located within the pocket, recess or bore 21 is positioned or located approximately 0.3 mm below the surface of the well bottom. Therefore, according to this embodiment reagent beads or microspheres 20A located and secured in the pockets, recesses or bores 21 provided in the bottom of a sample well 19 do not project above the entrance to or surface of the pocket, recess or bore 21 and hence do not project above the bottom surface of the sample well 19. However, according to other embodiments one or more reagent beads or microspheres may be located in one or more pockets, recesses or bores 21 provided in the base of the sample well 19 and may be located in relatively shallow pockets, recesses or bores 21 or may be located in one or more pockets, recesses or bores 21 which have a taper such that when the reagent bead or microsphere 20A is securely positioned or inserted within the pocket, recess or bore 21 then the reagent bead or microsphere projects above the entrance or surface of the pocket, recess or bore 21 and hence projects above the bottom surface of the sample well 19. The reagent beads or microspheres 20A may be arranged such that they protrude 20-40% of their diameter above the bottom surface of the sample well.

Reagent beads or microspheres may be dispensed or inserted into pockets, recesses or bores 21 provided in the bottom of a sample well 19 of a sample plate by means of a reagent bead or microsphere dispenser or inserter.

### Overview of Microarrayer Apparatus

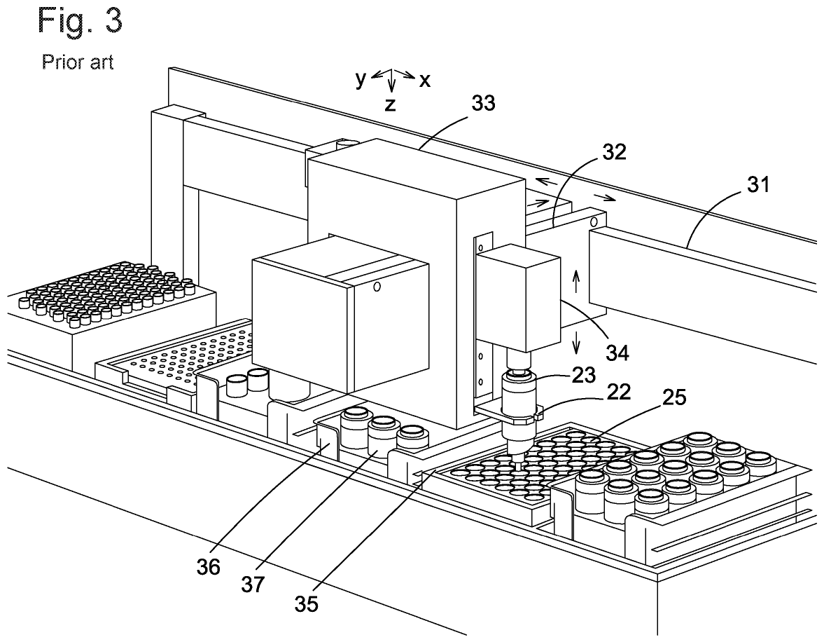

A microarrayer or automated apparatus is shown in FIG. 3 and may comprise a plurality of syringe bodies 37 loaded onto a tray or pack 36 which is then automatically loaded into the microarrayer or automated apparatus. The tray or pack 36 comprises a plurality of syringe bodies 37 and may be moved by a three-axis translation mechanism or robotic arm to a reagent bead or microsphere dispensing work area of the microarrayer or automated apparatus.

The microarrayer or automated apparatus may comprise a three-axis translation mechanism which may comprise a first translation stage comprising a guide rail 31 along which a first arm 32 may be translated in a first (x) horizontal direction. A second translation stage is preferably provided and comprises a mounting block 33 which preferably encompasses or surrounds the first arm 32. The mounting block 33 may be translated in a second (y) horizontal direction (which is preferably orthogonal to the first (x) horizontal direction) and may be moved backwards and forwards along the first arm 32. A third translation stage is preferably provided and may comprise a body or syringe drive mechanism 34 which preferably houses a linear actuator (not shown). The body or syringe drive mechanism 34 is preferably slidably mounted on the mounting block 33 and may be raised and lowered in a vertical (z) direction.

The three-axis translation mechanism preferably further comprises a retractable arm 22 which preferably extends from the mounting block 33. The three-axis translation mechanism is preferably programmed to select and pick up a reagent bead or microsphere dispenser 37 from the tray or pack 36 comprising a plurality of reagent bead or microsphere dispensers 37. The body or syringe drive mechanism 34 comprises a tapered spigot which is resiliently mounted within a tubular housing. The spigot is arranged to engage with a tapered portion provided on the syringe cap 23 of the reagent bead or microsphere dispenser 37. When a reagent bead or microsphere dispenser 37 is positioned in the tray or pack 36 the spigot may be lowered onto the syringe cap 23 of a reagent bead or microsphere dispenser 37 thereby securing the reagent bead or microsphere dispenser 37 to the body or syringe drive mechanism 34 in a detachable manner. The body or syringe drive mechanism 34 and attached reagent bead or microsphere dispenser 37 may then be raised to a height such that the retractable arm 22 (which is initially retracted within the body of the mounting block 33) can then be extended. The reagent bead or microsphere dispenser 37 is then lowered by the body or syringe drive mechanism 34 so that the upper portion of the syringe body is secured by the retractable arm 22. The retractable arm 22 preferably has an aperture having an internal diameter which is preferably smaller than the outermost diameter of a rim of the upper portion of the syringe body.

Each reagent bead or microsphere dispenser 37 may comprise a plurality of identical reagent beads or microspheres. According to an embodiment up to 15 separate reagent bead or microsphere dispensers 37 may be loaded or provided in a single tray or pack 36 and each of the reagent bead or microsphere dispensers 37 may have a capacity of up to approximately 2000 reagent beads or microspheres.

The syringe drive mechanism 34 may be arranged to pick a reagent bead or microsphere dispenser 37 out of the tray or pack 36 and will position and lower the barrel of the reagent bead or microsphere dispenser 37 so that it is immediately above a desired reagent bead or microsphere pocket or recess 21 provided in a sample well 19 of a sample plate. The syringe drive mechanism 34 may then be actuated so that the actuator or plunger boss of the reagent bead or microsphere dispenser 37 is depressed which in turn causes the plunger to push a reagent bead or microsphere from the chamber through a silicone member, through a barrel and into a desired reagent bead or microsphere pocket or recess 21 of the sample well 19. The syringe drive mechanism 34 may be arranged to depress the actuator boss and plunger with a desired amount of force as opposed to moving the actuator or plunger boss and plunger to a certain vertical position. As a result, reagent beads or microspheres are pressed-in tightly and consistently into the reagent bead or microsphere pockets or recesses 21 of a sample well 19 with a constant amount of force.

A test was performed wherein a sample plate comprising nine sample wells 19 was provided. Each sample well 19 comprised ten pockets, recesses or bores 21 which were arranged in a circle around a central portion of the sample well 19. Each of the pockets, recesses or bores 21 were loaded with reagent beads or microspheres which were coated with different concentrations of reagent. The ten beads in the first sample well were coated with a reagent having a concentration of 10 μg/ml and the ten beads in the second sample well were coated with a reagent having a concentration of 8 μg/ml. The ten beads in the third sample well were coated with a reagent having a concentration of 4 μg/ml and the ten beads in the fourth sample well were coated with a reagent having a concentration of 2 μg/ml. The ten beads in the fifth sample well were coated with a reagent having a concentration of 1 μg/ml and the ten beads in the sixth sample well were coated with a reagent having a concentration of 0.5 μg/ml. The ten beads in the seventh sample well were not coated with a reagent i.e. the concentration was 0 μg/ml. The ten beads in the eighth sample well were coated with different concentrations of reagent and comprised concentrations of 10 μg/ml, 8 μg/ml, 4 μg/ml, 2 μg/ml, 1 μg/ml, 0.5 μg/ml, 0 μg/ml, 0 μg/ml, 0 μg/ml and 0 μg/ml. The ten beads in the ninth sample well had the same concentrations as the reagent beads or microspheres in the eighth sample well and were arranged in the same manner as the reagent beads or microspheres in the eighth sample well.

The reagent beads or microspheres were coated with a capture antibody comprising sheep IgG and were transported in a bicarbonate buffer containing 0.02% Kathon® preservative.

The sample wells 19 of the sample plate were emptied of the preservative in which the reagent beads or microspheres were transported in and 400 μl of a 1/1000 diluted donkey anti-sheep IgG peroxidise conjugate in a Tris Buffered Saline (“TBS”) conjugate diluent buffer was added to each sample well 19. The sample plate was then incubated at ambient temperature and was subjected to medium intensity vibrations for a period of 45 minutes. Any unbound conjugate was then aspirated from the sample wells 19 using a single channel wash head of a microarrayer apparatus (DS2®, available from Dynex Technologies®). Once any unbound conjugate had been aspirated from the sample wells 19, 500 μl of 1/20 diluted Tris Buffered Saline wash fluid was then immediately added to each sample well 19. The wash fluid was then aspirated from the sample wells 19 and the process of washing and aspirating wash fluid from the sample wells 19 was repeated twice more. After the third washing step including aspiration of wash fluid had been completed, 300 μl of luminol (a chemiluminescent marker) was then immediately added to each sample well 19. The sample plate was then incubated in the dark at ambient temperature whilst being subjected to medium intensity vibrations for 15 minutes. The sample plate was then transferred immediately to a reading chamber.

A camera was set to an exposure time of 6 minutes and 30 seconds with a gain of ×20. Images were taken at 22 minutes and 29 minutes after luminol had been added. The camera exposure time was then changed to 8 minutes and 37 seconds. Further images were taken at 38 minutes, 47 minutes, 56 minutes and 65 minutes after luminol addition. Analysis of the images showed that the greatest observed signal strength was obtained after 15-22 minutes from luminol addition which is consistent with the luminol decay curve.

The following steps may be carried out once reagent beads or microspheres have been dispensed or inserted into pockets, recesses or bores of a sample plate. Firstly, sample fluid may be added to one or more sample wells of the sample plate. The sample fluid may comprise one or more analytes such as specific antigens which may react with reagent coated on one or more of the reagent beads or microspheres. The reagent beads or microspheres are preferably coated with a specific capture antibody.

Once the sample fluid has been added to the sample wells, the sample plate is then preferably subjected to an incubation step. After the sample plate has been subjected to an incubation step so that antigen-antibody complexes are formed, the sample plate is then preferably subjected to one or more washing and aspirate steps in order to remove any unbound sample fluid and to remove any wash fluid. An enzyme conjugate is then added which will bind to the antigen part of any antigen-antibody complexes which have been formed but which will not bind to antibodies or to the antibody part of an antigen-antibody complex. The sample plate is then incubated before being subjected to one or more washing and aspirate steps. Once the sample plate has been subjected to one or more washing and aspirate steps luminol (or another visualising agent) is preferably added. The sample plate is then preferably aspirated to remove any excess luminol (or other visualising agent). The luminol (or other visualising agent) upon contacting enzymes attached to the antigen part of an antigen-antibody complex will then breakdown causing a distinctive colour to be produced. In the final stage the sample plate is analysed and an endpoint determination is preferably made.

### Conventional Sample Plate

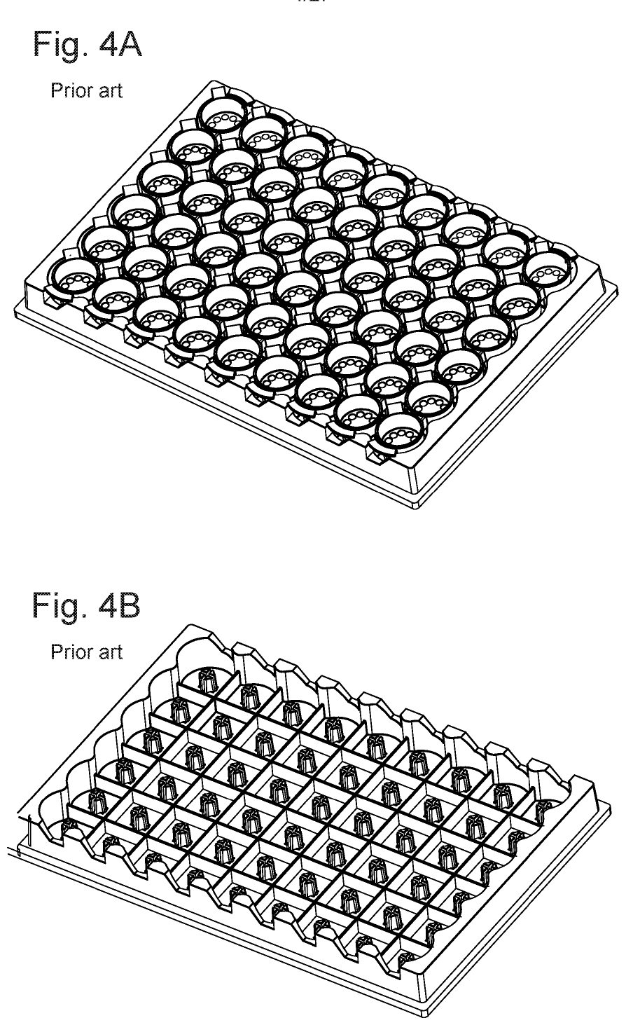

FIG. 4A shows nine sample strips loaded into a plate frame. Each of the sample strips shown in FIG. 4A comprises a 6×1 strip of sample wells. The sample strips can be removeably loaded into the plate frame. Each of the nine sample strips comprises six sample wells and each sample well may comprise ten (optionally tapered) bores which, in use, are arranged to receive a reagent bead. The reagent beads are preferably loaded or pre-loaded into the bores such that the reagent beads protrude above the base portion of the sample well. FIG. 4B shows in more detail the plate frame into which the sample plates may be loaded.

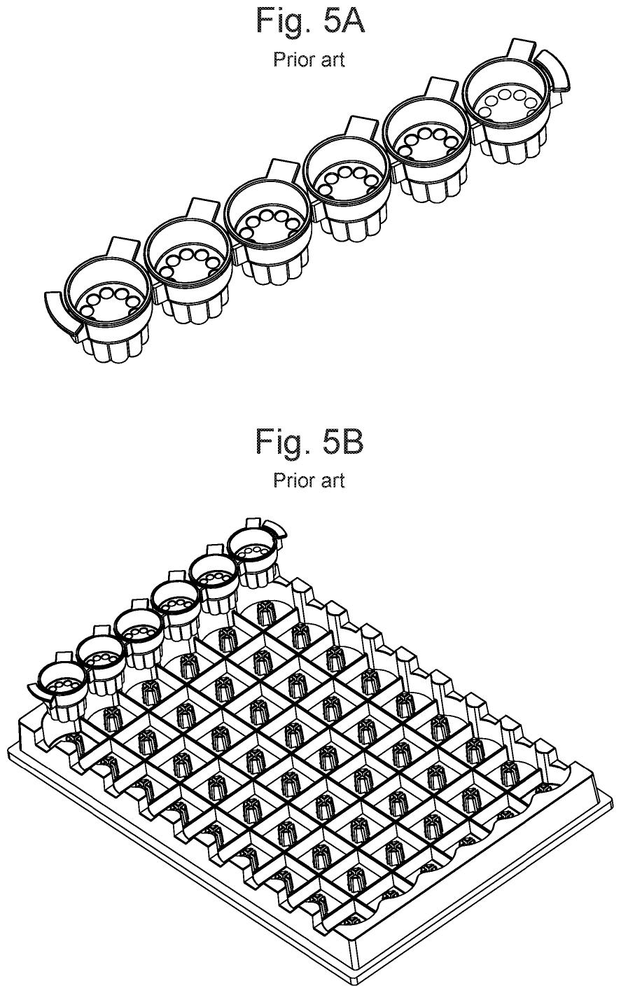

FIG. 5A shows in greater detail a sample strip comprising six sample wells. The sample wells in a strip can be separated or otherwise broken apart. According to an embodiment the sample plate or strip can be separated or divided up into single sample wells. FIG. 5B shows a sample strip comprising six sample wells being loaded into a plate frame.

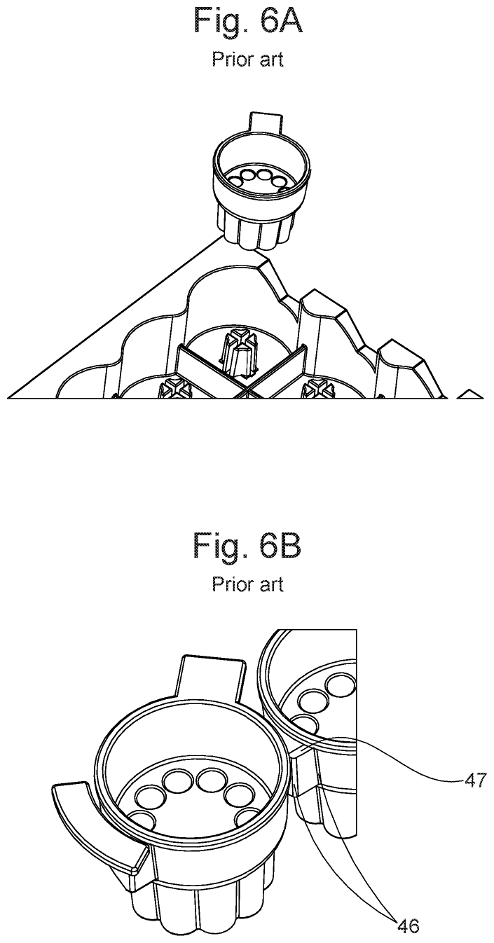

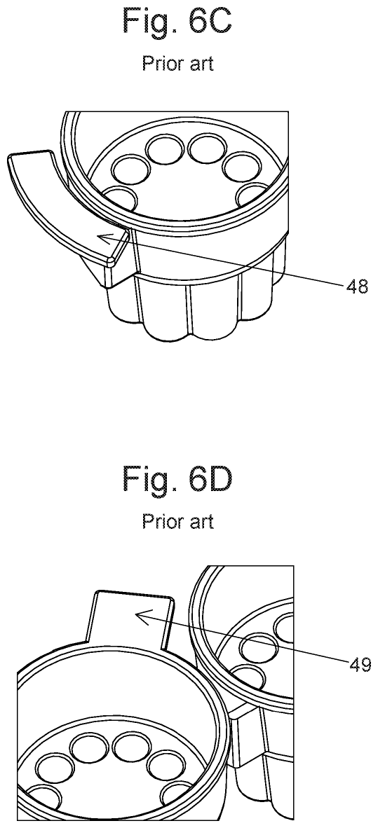

FIG. 6A shows a single sample well (which has been separated from a strip of sample wells) being loaded into a plate frame. The sample wells preferably comprise a female portion which is preferably arranged to engage or interlock with a male portion which is preferably provided on the base of the plate frame. The sample plate or sample strip is preferably arranged to be firmly secured and fixed to the plate frame when loaded onto the plate frame.

FIG. 6B shows in greater detail two sample wells which are connected by a break-apart feature 47. The break-apart feature 47 preferably allows a user to separate adjacent sample wells. According to an embodiment sample wells may be separated from each other but may still be placed next to each other on the plate frame without interfering with each other. The break-apart feature 47 may comprise one, two or more than two break points 46. According to an embodiment the connecting piece 47 between two sample wells may be separated from a sample well at a first break point 46. The connecting piece 47 may then be broken off or otherwise removed from the single sample well that it is attached to by breaking the connecting piece 47 from the sample well at a second break point 46.

FIG. 6C shows a sample well having an end break-apart feature 48. The end break-apart feature 48 allows the end wells to be used singly in the plate frame without interfering with another sample well. The end break-apart feature 48 provides something for a user to hold in order to remove a strip of sample wells or a single sample well from the plate frame.

FIG. 6D shows a sample well having an ID and orientation tab 49. The tab 49 allows an identifier to be printed onto the tab 49 or to be otherwise attached to the tab 49. The identifier may comprise a 2D or 3D barcode and/or human readable text. The tab 49 preferably assists a user to orientate a sample well when a single sample well is used by aligning with features in the plate frame and/or on other sample wells.

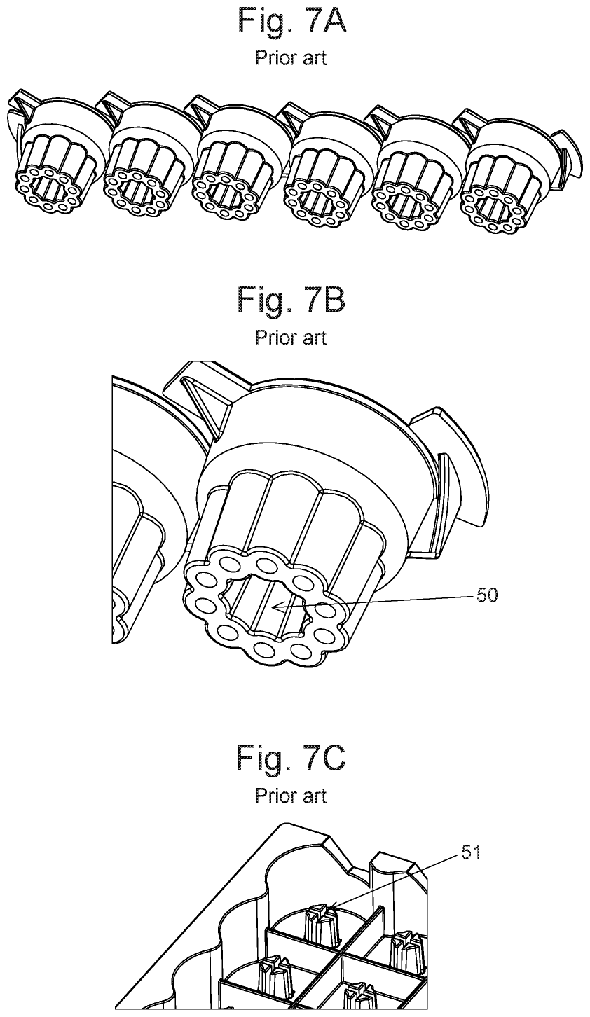

FIG. 7A shows the underneath of a strip of sample wells and shows that each sample well comprises ten bores or recesses in which a reagent bead is preferably inserted in use. The base or underside of each sample well preferably also comprises a female portion which is preferably arranged to be mated, in use, with a male portion which is provided in the base of the plate frame.

FIG. 7B shows in greater detail a female alignment and retaining feature 50 which helps to align a strip of sample wells with a plate frame. FIG. 7C shows a corresponding male alignment and retaining feature 51 which is preferably provided in the base of the plate frame. The male portion 51 may according to an embodiment comprise a plurality of flexible projections which are preferably deformed inwards as a sample well is located over the male portion 51. The projections on the plate frame preferably move or close together ensuring that the sample well is kept in place without having to apply undue force either to mount or fix a sample well onto the plate frame and/or to demount a sample well from the plate frame.

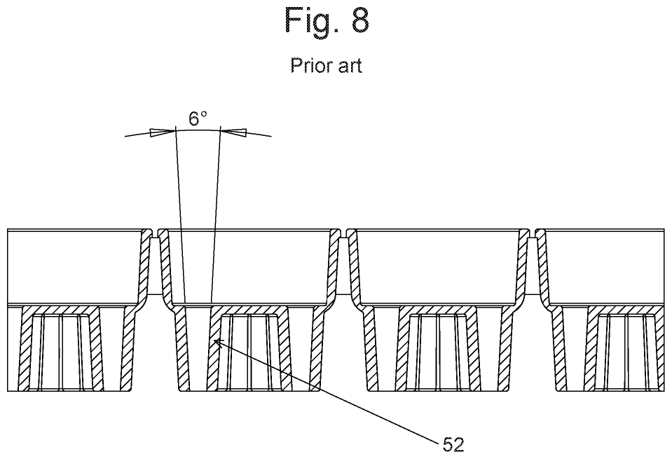

FIG. 8 shows a cross-sectional view of a strip of sample wells and shows that the sample wells may comprise a plurality of tapered bores 52. The tapered bores 52 act as pockets into which a reagent bead is inserted in use. The angle of the taper in the arrangement shown in FIG. 8 is 6.0°.

Although various arrangements described above have focussed upon reagent beads which are coated with a biomolecule for use in an Immunoassay or ELISA procedure, the present invention equally applies to reagent beads which comprise or which are otherwise coated with a nucleic acid sequence and which are used as a hybridization probe for the detection of DNA or RNA sequences which are complementary to those provided on the reagent beads. As will be understood by those skilled in the art, the hybridization probe will be inactive until hybridization, at which point there is a conformational change and the molecule complex becomes active and will then fluoresce under UV light. Therefore, all the various embodiments described above and all the various aspects of the embodiments described above apply equally to the use of reagent beads comprising or which are otherwise coated with a DNA or RNA sequence (or other nucleotide sequence) for use as a hybridization probe to detect complementary DNA or RNA sequences.

Many variants, including fluorogenic and luminogenic substrates for ELISA, direct labeling of the second member of the binding pair with a fluorescent or luminescent molecule (in which case the procedure is not called an ELISA but the process steps are very similar) and nucleic acids or other specific pairing agents instead of antibodies can be used as a probe. The same principles can be used to detect or determine any materials which can form specific binding pairs, for example using lectins, rheumatoid factor, protein A or nucleic acids as one of the binding partners.

The sample plate or multiplexed sample plate according to the present invention can thus be used to detect one or more analytes, such as one or more biomarker, which can be indicative of a disease or condition. The disease or condition can be a tumor, neoplasm, or cancer, such as breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The disease or condition can also be an inflammatory disease, immune disease, or autoimmune disease, such as inflammatory bowel disease (IBD), Crohn's disease (CD), ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis. The disease or condition can also be a cardiovascular disease, such as atherosclerosis, congestive heart failure, vulnerable plaque, stroke, ischemia, high blood pressure, stenosis, vessel occlusion or a thrombotic event. The disease or condition can also be a neurological disease, such as Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neurospsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The phenotype may also be a condition such as fibromyalgia, chronic neuropathic pain, or peripheral neuropathic pain. The disease or condition can also be an infectious disease, such as a bacterial, viral or yeast infection. For example, the disease or condition may be Whipple's Disease, Prion Disease, cirrhosis, methicillin-resistant Staphylococcus aureus, HIV, hepatitis, syphilis, meningitis, malaria, tuberculosis, or influenza. Viral proteins, such as HIV or HCV-like particles can be assessed in an exosome, to characterize a viral condition.

The sample plate or multiplexed sample plate can be used to detect one or more biomarkers, biomolecules or analytes that are used to detect the disease or condition. For example, the detection of a biomarker can be used to detect or provide a diagnosis, prognosis of a disease or condition. For example, the sample plate or multiplexed sample plate can comprise one or more probes for one or more cancer markers and may be used to detect one or more cancer markers in a sample from an individual. The presence, absence, or level of a cancer marker in the sample can be indicative of cancer in the individual. In another embodiment, the sample plate or multiplexed sample plate may be used to monitor a disease or condition. For example, an increased level of one or more cancer markers, as compared to a control, or compared to an earlier assay for the one or more cancer markers from the same individual, may be indicative of progression of the cancer. In yet another embodiment, the sample plate or multiplexed sample plate can be used to in determine a therapy or course of action for a condition. For example, an individual may have a genetic variant which leads to the individual being unable to metabolize certain drugs. The sample plate or multiplexed sample plate can be used to detect the genetic variant. In another embodiment, the sample plate or multiplexed sample plate may be used to detect a compound, which can be indicative of a drug not being metabolized. The sample plate or multiplexed sample plate can also be used to detect the intake of certain drugs or compounds, such as by detecting a drug or by-products of a drug, which can be used for drug testing.

The sample plate or multiplexed sample plate can also be used to screen for drugs. For example, the sample plate or multiplexed sample plate can comprise one or more probes that are target(s) for drug development. The sample plate or multiplexed sample plate can then be used to screen a library of compounds. Alternatively, the sample plate or multiplexed sample plate can comprise a plurality of probes that comprise a library of compounds that are potential drugs. The sample can comprise a drug target, which is added to the sample plate.

Also provided herein is a kit comprising a sample plate or multiplexed sample plate disclosed herein. The kit can comprise one or more components for detecting an analyte or for performing an assay. In one embodiment, a kit for detecting an analyte comprises one or more sample plates and a plurality of beads, plugs or inserts. The plurality of beads, plugs or inserts can comprise one or more probes, such as a probe that is a nucleic acid, antibody, antibody fragment, protein, peptide, aptamer, or chemical compound. In another embodiment, a kit for performing an Enzyme Linked ImmunoSorbent Assay (ELISA) procedure is provided. The kit can comprise one or more sample plates or multiplexed sample plates as described herein; and a plurality of beads, plugs or inserts wherein the beads, plugs or inserts are coated with a reagent comprising an antibody, an antigen or another biomolecule. In yet another embodiment, the kit can comprise components for performing a nucleic acid probe procedure, wherein the kit comprises one or more sample plates or multiplexed sample plates as described herein; and a plurality of beads, plugs or inserts coated with a nucleic acid, such as a DNA or RNA probe or sequence.

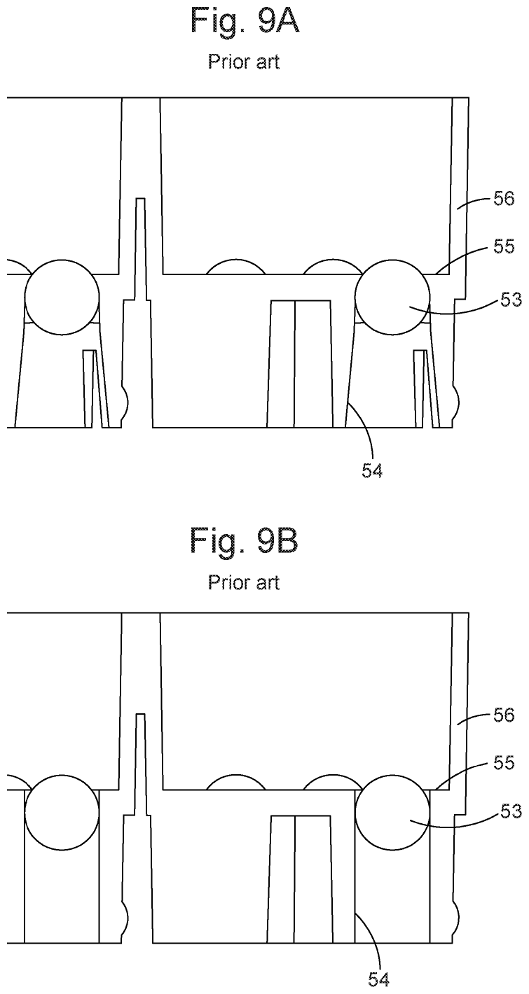

FIG. 9A shows how reagent beads 53 may be loaded into a sample plate from the underneath or rear side of the sample plate. The sample plate may comprise a bore or through hole 54 which according to the arrangement shown in FIG. 9A is tapered. However, as will be discussed below, it is also contemplated that the bore or through hole may not be tapered and may instead comprise a substantially cylindrical through hole or bore 54 which has a substantially constant cross-sectional diameter and/or area and/or profile. FIG. 9B shows a sample plate wherein reagent beads or microspheres are secured within a cylindrical bore or through hole 54. The reagent beads or microspheres may be inserted into the cylindrical bore or through hole 54 either from the top or from the bottom. The reagent beads or microspheres are preferably secured within the bore or through hole 54 by an interference fit and the reagent beads or microspheres make a substantially fluid-tight seal around a full circumference of, perimeter of or closed loop around the reagent bead or microsphere.

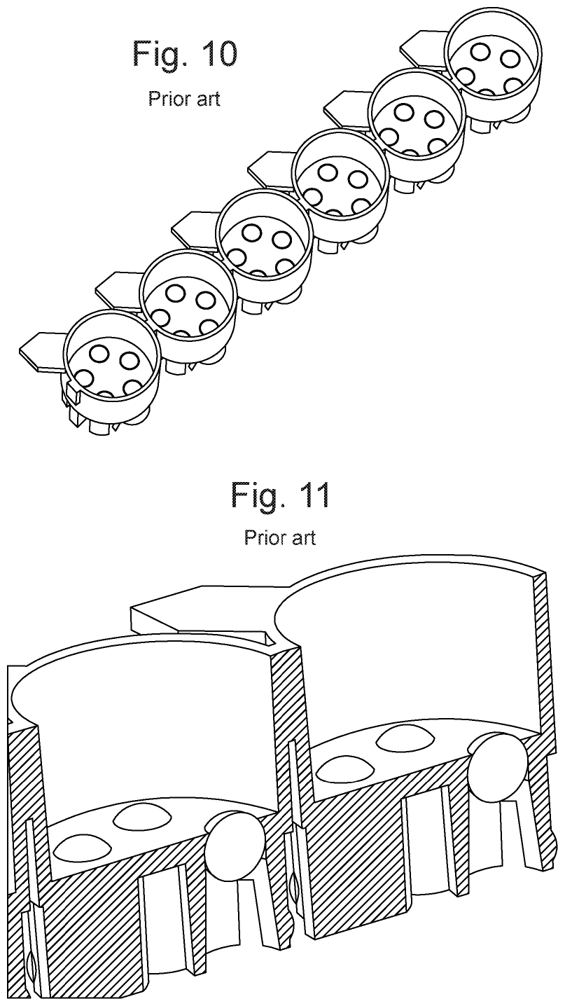

With regard to the arrangement shown in FIG. 10 and referring back to FIG. 9A, bores or through holes 54 in a sample well may taper from a first diameter at the lowermost part or bottom of the base portion 55 of the sample well 56 to a second narrower diameter towards the uppermost part or top of the base portion 55. The uppermost part or top of the base portion 55 is that part of the base portion 55 which preferably comes into contact with sample fluid in use.

At the top of the bore or through hole 54 immediately below the portion of the base portion 55 which comes into contact with sample fluid, the bore or through hole 54 may be shaped so as to form a tight fit with a reagent bead 53. The uppermost portion of the bore or through hole may comprise a part spherical profile, bulbous region, curved portion or concave region so that a reagent bead 53 which is inserted into the bore or through hole 54 from the underneath of the sample plate fits tightly within the part spherical profile, bulbous region, curved portion or concave region at the top of the bore or through hole 54 as shown in FIG. 9A.

A portion of the reagent bead 53 projects into the base or bottom of the sample well to form, in effect, part of the base portion of the sample well 56. As a result, the top portion of the reagent bead 53 (above the region where the bead forms a fluid-tight circumferential seal with the wall of the through hole) is arranged so as to come into contact with sample fluid in use. The reagent bead 53 forms a fluid tight seal around the full circumference of the bead 53 with the part spherical profile, bulbous region, curved portion or concave region of the bore or through hole 54.

Macro sized beads 53 may be fitted into a sample well 56 of a sample plate so that only the top or upper portion of the reagent bead 53 is exposed to fluid. It should be noted that the luminescent reading process is a 2D operation and only takes into account signal from the visible portion of the reagent bead 53 facing the camera. As will be discussed in more detail below, having reagent beads project into the bottom of the sample well can cause problems due to crosstalk and due to the creation of dead zones if the sample well is agitated.

The multiplex well together with reagent beads loaded into the through holes preferably mimics the well established microplate ELISA type of process. The multiplex well may be substantially similar in format to a microplate well.

One of the major factors in processing an ELISA test in a microplate is the efficiency or cleanliness of each step. Any residual fluid from the steps can have an overall effect on the performance of the test e.g. if the conjugate is not completely removed by washing, then residual conjugate will produce a false signal on the bead. This will drive down the sensitivity of the test by increasing the background signal.

The key to efficient processing of the test is not to have any fluid traps in the well. Any corners, pockets or undercuts may trap fluid thereby reducing the performance of the sample plate. The sample plate allows efficient washing, mixing and aspirating in a similar manner to a conventional microplate well and preferably does not suffer from the problem of trapping fluid.

Beads 53 are fitted at a uniform height in a sample well 56 which preferably ensures that each bead 53 is treated identically. Each bead 53 makes a fluid tight sealed fit in the locating detail of a pocket of through hole to ensure that there is no fluid trapped under or below the bead 53.

The through hole 54 may comprise a tapered conical hole in which the bead locks into the hole as shown in FIG. 9A or the through hole 54 may comprise a cylindrical undersized hole into which a bead is mechanically pressed into as shown in FIG. 9B. Both arrangements achieve the goal of preventing fluid going past the bead 53 and becoming trapped underneath or below the bead 53.

If the sample plate comprises one or more tapered through holes 54 as shown in FIG. 9A then the through holes may be manufactured with a high degree of accuracy and consistency to ensure that beads are secured within the sample plate at a uniform height (since the reagent beads 53 are preferably pressed into the through holes 54 with a set force and not to a set height). The alternative arrangement of using undersized cylindrical through holes as shown in FIG. 9B does not need to be manufactured to so such a high degree of accuracy since the reagent beads 53 are pressed in to the through holes to a set height and not with a set force.

In some of the arrangements described above reagent beads may be fitted into a blind pocket detail in a sample well i.e. into a closed recess. However, more preferably, a sample plate having through holes in the base portion may be provided as shown and described above with reference to FIGS. 9A and 9B.

The assembly of a sample plate or multiplexed sample plate which is loaded with reagent beads during production or manufacture may be subjected to a quality control check to ensure that all the beads are sealed to the sample plate or multiplexed sample plate. Beads which are loaded into blind pockets as described above will ensure that fluid will not leak out of the well. However, fluid might still leak under the bead and such a leak would be difficult to detect.

A sample plate or multiplexed sample plate comprising through holes as shown in FIGS. 9A and 9B allows a pressure check to be carried out as part of the bead to plate assembly, manufacture and quality control checks. This ensures that the bead to plate seal is good. A defective bead or damaged hole would show up as a fail in the manufacture and not when the user runs the test.

The sample plate according to the arrangements as shown in FIG. 9A or optionally also in FIG. 9B wherein reagent beads are fitted into the bore from underneath is particularly advantageous for a number of reasons. Firstly, contact between a press-in tool and the bead 53 is with the bottom or underneath portion of the reagent bead 53 so any witness mark will also be on the bottom or underneath portion of the reagent bead 53 i.e. not any portion of the reagent bead 53 which will come into contact with sample fluid. Secondly, the top of the through hole 53 in the base portion 55 of the sample well in the example shown in FIG. 9A can be made to match the profile or shape of the reagent bead 53 so that no moat portion is formed around the portion of the bead 53 which protrudes into the base of the sample plate. As a result, the design excludes any possibility of trapping fluid below the reagent bead 53. Thirdly, it does not matter if the tip of a press-in tool effectively cross contaminates other beads since the press-in tool will only come into contact with the underneath or bottom portion of the reagent beads 53. The press-in tool does not come into contact with the top portion of the reagent beads 53 (i.e. the portion of the reagent beads 53 which will come into contact with sample fluid). Fourthly, in the embodiment shown in FIG. 9A reagent beads 53 can be fitted lower in the base portion without forming a moat region and in a manner which reduces the risk of crosstalk.

A system for preparing arrays of biomolecules is disclosed in US2009/0069200. FIGS. 2 and 3 of US2009/0069200 show spherical reagent beads 9 located in square subwells 8. It is apparent, therefore, that the circular beads placed in the square subwells do not make a fluid-tight seal with the walls of the subwells. The arrangement disclosed in US2009/0069200 also differs from the disclosed arrangement in that fluid is arranged to pass up through sub wells and over the beads. In contrast, according to the disclosed arrangement fluid is only arranged to come into contact with the top surface of a reagent bead 53. Fluid is prevented from passing down a through hole 54 or recess past a reagent bead 53 secured within the through hole 54 or recess.

Advantageously, a sample plate according to the disclosed arrangement can be cleaned easily during the process steps without trapping fluid under the reagent beads 53. The beads 53 are preferably provided in a format that makes it as close to a cylindrical well as possible and which can also be easily accessed from the top.

The arrangement disclosed in US2009/0069200 uses a common filling chamber or reservoir beneath the beads that is dispensed into in order for the fluid to rise up the individual wells. Circular beads are lodged in square tapered sub wells i.e. the beads do not make a fluid tight seal with the sub wells. Indeed, the fact that spherical beads are provided in square wells enables fluid to flow up, past and around the beads.

The sample plate as disclosed in US2009/0069200 would need to be manufactured in two separate parts as it would not be possible to mould the sample plate including a reservoir as a single piece. The lower part of the sample plate is shown as comprising a discrete plate bottom 11 which would need to be sealed to the upper section of the sample plate comprising a plurality of wells 7 during the manufacturing process. Each well 7 has to be sealed to the plate bottom 11 to ensure that it does not leak. Therefore, the entire grid face between the lower plate bottom 11 and the upper sample wells 7 has to be sealed reliably. As a result, the manufacture process is relatively complex and prone to manufacturing problems.

The sample plate as disclosed in US2009/0069200 is also particularly complex in respect of fluid flow dynamics. The initial dispensing of fluid into the sample plate has to be carried out by dispensing through one of the sub wells. As a result, fluid must be accurately dispensed into a small target area <1.7 mm which is substantially smaller than the diameter of a sample well. Furthermore, once fluid has been dispensed into one of the sub wells then the fluid has to flow into the chamber or reservoir 12 at the bottom of the sample plate before rising up evenly into each of the wells to ensure that all the beads are sufficiently immersed. It will be appreciated, therefore, the fluid dynamics associated with the arrangement disclosed in US2009/0069200 are complex and involve tortuous paths which does not lend itself to reproducible results.

Once the sample or conjugate fluid has been dispensed and has flowed past or over the beads in the arrangement disclosed in US2009/0069200, the fluid must then somehow be removed in a commercial product. However, this is particularly problematic as the only access to the sample plate is from the top. Even if a rectangular vacuum tube were sealed against the top of a well it could not be guaranteed that all fluid in the chamber or reservoir in the bottom of the sample plate would be removed. As a result, it is likely that some fluid residue would be left behind in the reservoir and which could cause a false signal in the well.

It will be appreciated, therefore, that the arrangement disclosed in US2009/0069200 suffers from a number of significant problems.

In contrast, the sample plate according to the disclosed arrangements does not suffer from the above mentioned problems and represents a significant improvement over known arrangements such as that disclosed in US2009/0069200.

FIG. 10 shows a strip of six sample wells with five 3 mm reagent beads loaded into through holes in each sample well. The reagent beads are loaded into the through holes from the bottom or underneath of the sample plate. The reagent beads are retained within the through holes by upper concave regions formed in the through holes.

FIG. 11 shows a three dimensional cross-sectional view of the arrangement as shown and described above with reference to FIGS. 9A and 10.

### Reagent Bead, Plug or Insert Inserter

A reagent bead, plug or insert inserter may be used to insert reagent beads, macrobeads, plugs or inserts into one or more bores of a sample plate or multiplexed sample plate.

The disclosed sample plate or multiplexed sample plate enables multiple tests to be carried out in a single well of a sample plate or multiplexed sample plate. The technology may use macro sized (e.g. mm sized) reagent beads, plugs or inserts that are coated with specific antigens or antibodies. Each well of a sample plate or multiplexed sample plate comprises multiple bores in the base portion of the sample plate or multiplexed sample plate. Reagent beads, macro beads, plugs or inserts may be pressed and retained in the bores of each well by an interference fit so that the top of a reagent bead, plug or insert is exposed to the assay test.

U.S. Pat. No. 6,074,609 discloses a system for arraying microbeads. The microbeads disclosed in U.S. Pat. No. 6,074,609 are of the order of 5-300 μm i.e. are an order of magnitude smaller than the macrobeads used according to the disclosed arrangement. The microbeads are stored in a reservoir holding a liquid medium. A distal end of a transfer member is lowered into the liquid medium and a vacuum is created within a lumen to draw a microbead onto the distal end of the transfer member. The transfer member is then lifted from the reservoir whilst holding the microbead on the distal end. The transfer member is then positioned in a test well holding another liquid medium. The vacuum is then removed and the microbead is released from the transfer member whilst the transfer member is within the liquid medium. The microbead is then allowed to fall under the force of gravity within the liquid medium.

There are a number of problems with the arrangement disclosed in U.S. Pat. No. 6,074,609. One problem with the arrangement disclosed in U.S. Pat. No. 6,074,609 is that as a microbead is being drawn towards the distal end of the transfer member the lumen will at least partially fill with fluid. This can cause a serious problem with cross-contamination.

Another problem with the arrangement disclosed in U.S. Pat. No. 6,074,609 is that the microbeads and in particular any sensitive coating on the microbeads may become damaged whilst the microbead is being transferred by the transfer member.

It is desired to mass produce sample plates and to improve the process of locating reagent or macrobeads in the bores of a sample plate.

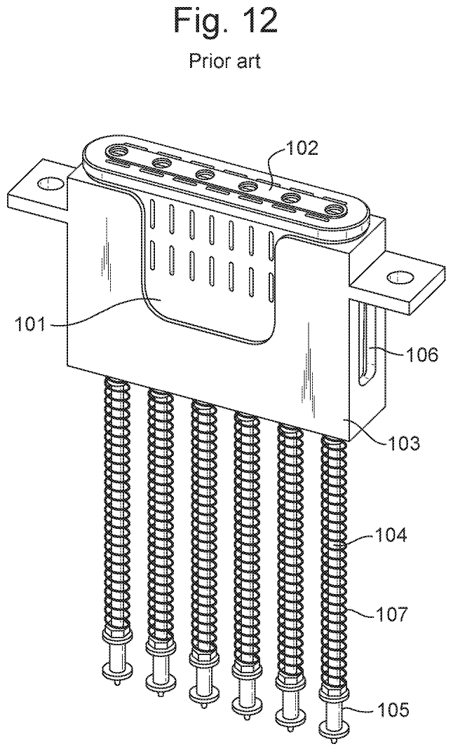

With reference to FIG. 12 a reagent bead inserter is disclosed wherein, reagent beads or macrobeads may loaded into one or more cartridges 101 by an operator and may be stored or placed directly on to a reagent bead insertion device. The operator may remove an upper cap 102 from the cartridge 101, pouring beads into the cartridge 101 and then replacing the cap 102. Alternatively, a manufacturer may supply a pre-loaded cartridge 101.

The upper cap 102 may comprise one or more apertures. The operator may apply a strip of tape or another closure device to some or all of the apertures in the cap 102 in order to prevent reagent beads from falling out of the cartridge 101. Alternatively, holes in the cap 102 may have silicone membranes which prevent beads from falling out.

The operator may apply a barcode label identification on to an end of the cartridge 101 or the cartridge may be supplied by a manufacturer with a barcode label identification. The operator then loads the filled cartridge 101 into a cartridge holder 103. The cartridge holder 103 may be positioned adjacent the insertion device. Alternatively, the cartridge holder 103 may be located distal to the insertion device and the cartridge holder 103 may be manually or automatically positioned adjacent the insertion device. An aperture or inspection window 106 is preferably provided in the cartridge holder 103 and enables a barcode label on the cartridge 101 to be inspected.

The bead insertion device may comprise a plurality of push rods 104 which are arranged so as to engage a lift drive mechanism at a lower end. The bottom or lower ends of the push rods 104 preferably each comprise a connection boss 105. The connection bosses are held securely in the lift drive mechanism so that the push rods 104 are subsequently positively driven linearly in an up and down direction. The bottom face of the connection bosses 105 is arranged to seal to the lift drive mechanism during engagement.

The push rods 104 comprise one or more axial bores which extend the whole length of the push rods 104. At the lower end of the push rods 104 the bore which extends through the connection bosses 105 preferably allows vacuum pressure to be routed through the push rods 104 to the end of the push rods 105. The vacuum or low pressure region which is created at the upper end of the push rods 104 is used to secure and retain a reagent bead or macrobead on the end of the push rod 104 during an insertion process.

At the base of the bead cartridge 101 one or more soft silicone membranes may be provided which allow the push rods 104 to enter the cartridge 101 without letting the beads fall out of the cartridge 101. As the push rods 104 travel up and through the bead cartridge 101 the push rods 104 each collect a reagent or macrobead on to the end of the push rod 104. The vacuum pressure sucks a single bead on to the end of each push rod 104 and retains the bead in a defined position on the end of the push rod 104.

The system is arranged to sense the change in vacuum pressure caused by a bead being sucked on to the end of a push rod 104 and sealing the open end of the push rod 104.

The push rods 104 continue to move up through the cartridge 1 and preferably extend out of the apertures in the cartridge cap 102. A sample plate or macroplate (not shown) is preferably positioned above the cartridge 101 so that specific well pockets or bores in the sample plate are aligned with the push rods 104 coming up through the cartridge 101 and exiting via the cartridge cap 102. The push rods 104 press reagent or macrobeads into bores formed within the sample plate or macroplate via the rear or lower surface of the sample plate or macroplate. The push rods 104 ensure that reagent or macrobeads are inserted into the bores of the sample plate at a desired height. Once reagent beads have been inserted or pressed into the bores of the sample wells, the insertion rods 104 are then driven in the reverse direction and return back down through the cartridge cap 102, the body of the cartridge 101 and the base of the cartridge 101. The push rods 104 are also returned to their initial position with the aid of push rod return springs 107. The system is preferably arranged and adapted to determine when reagent beads have been inserted into the bores in the wells of a sample plate and thus when the reagent beads have left the ends of the insertion rods by sensing changes in the vacuum pressure.

A cycle of inserting reagent beads into the sample wells of a sample plate is repeated one or more times until the sample plate or macroplate is loaded with a desired number of reagent or macrobeads of a first particular type. The system may comprise multiple cartridge holders 103 containing cartridges 101 each containing different specific bead types. The system may insert or fit all desired reagent beads of a first type and then disengage a cartridge holder 103 holding a cartridge 101 containing beads of the first type. The system may then engage a cartridge holder 103 holding a cartridge 101 containing beads of a second different type. The system may insert or fit all desired reagent beads of the second type into the sample plate. This process may be repeated with a third cartridge containing beads of a third different type and/or a cartridge containing beads of a fourth different type etc. until the sample plate is loaded with reagent beads of all desired types.

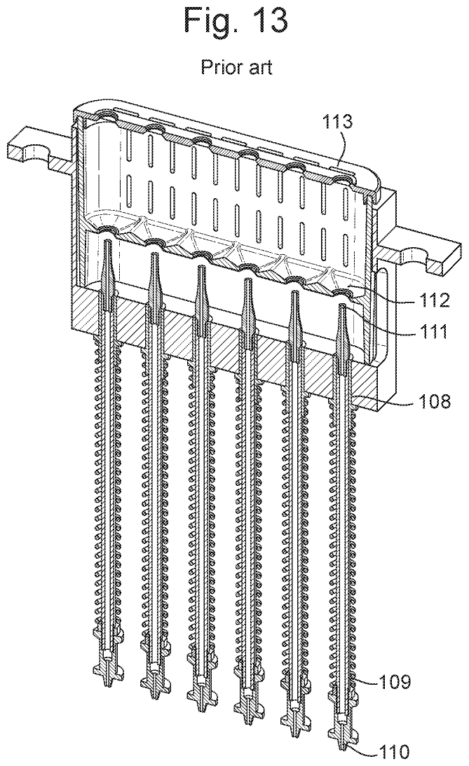

FIG. 13 shows a section through a cartridge holding assembly 103. The cartridge holder 103 may comprise a push rod guide bush 108. A push rod adjuster 109 is provided at one end of the push rods 104 together with a vacuum inlet 110. FIG. 13 shows the push rod ends 111 which have not yet entered the cartridge 101. The cartridge 101 may comprise an entry aperture 112 and a bead exit aperture 113. The entry aperture 112 is located on one (i.e. lower) side of the cartridge 101 and the bead exit aperture 113 is located on one opposite (i.e. upper) side of the cartridge 101.

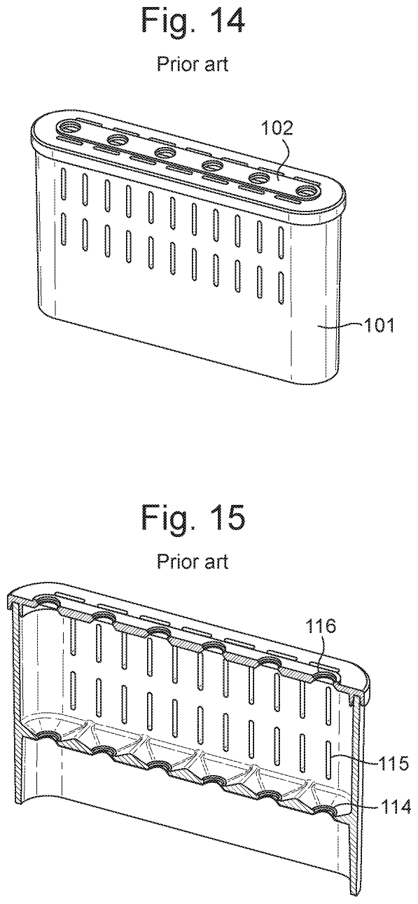

FIGS. 14 and 15 show a bead cartridge 101 which may comprise an injection moulded disposable housing. The assembly is made up of the cartridge body 101, a cartridge cap 102 having cap apertures 116 and a plurality of silicone membranes 114 provided around apertures in the base of the cartridge body 101. Optionally, a plurality of silicone membranes (not shown) may also be provided around the apertures 116 in the cartridge cap 102. The silicone membranes 114 are preferably moulded to the cartridge body 101 and/or the cartridge cap 102 using an over moulding process. One or more cartridge vents 115 may be provided in the housing of the cartridge 101.

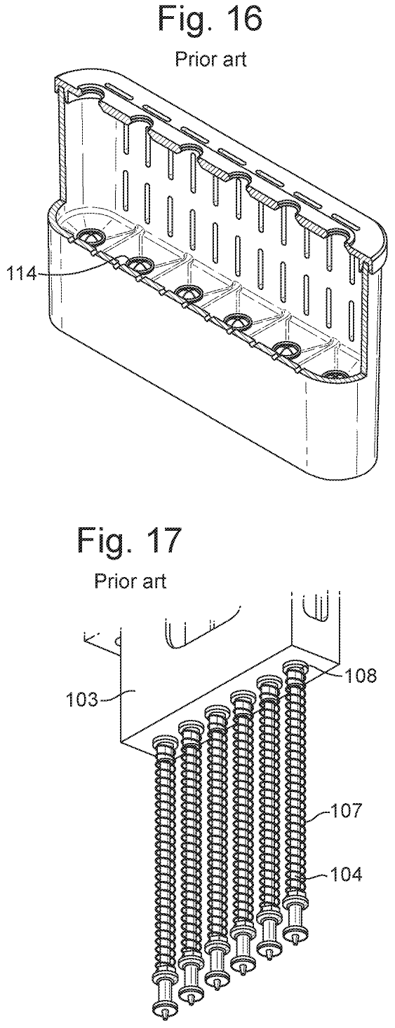

FIG. 16 shows a plurality of silicone membranes 114 provided around apertures in the base of the cartridge 101 in greater detail. The part of the membranes 114 that covers the holes or apertures in the base of the cartridge 1 preferably has cuts or slits moulded into it. The moulded cuts or slits may, for example, be in the shape of a cross allowing the membrane to fold out of the way when the push rods 104 travel through it. When the push rods 104 withdraw from the base of the cartridge 101, the membranes revert back to their original shape and prevent beads being pulled through the membrane and hence exiting the cartridge 101. The silicone membranes 114 are rigid enough to dislodge any beads inadvertently resting on the ends of the push rods 104.

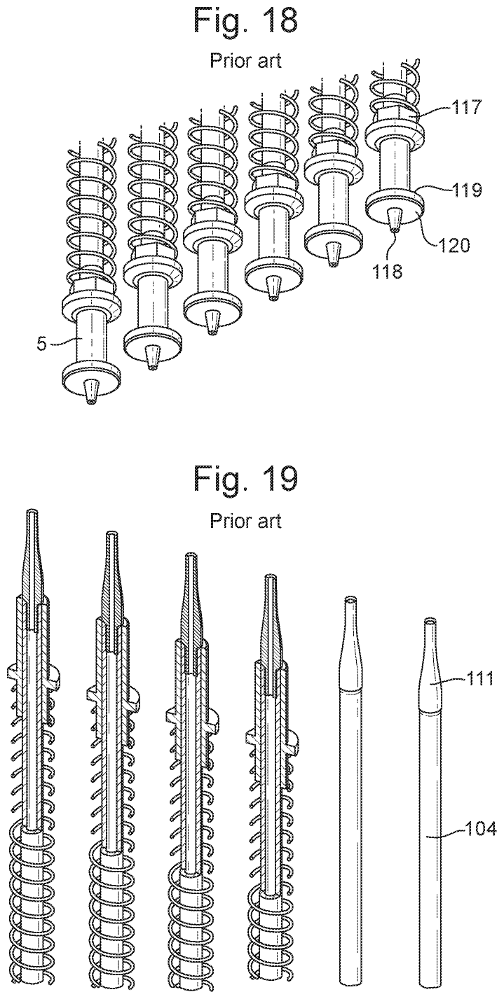

FIG. 18 shows the base of a cartridge holder assembly 103 in greater detail and shows six push rods 104 arranged to pass through the cartridge holder 103. However, other arrangements are contemplated wherein a different number of push rods 104 may be provided. In particular, eight push rods 104 may be provided. The push rods 104 slide in bearing bushes 108 located in a lower surface of the cartridge holder 103. The bearing bushes 108 ensure that the ends of the push rods 104 are located in the correct place relative to the macroplate or sample plate which is preferably arranged above the cartridge holder 103. Return springs 107 ensure that the push rods 104 are at the extent of their travel. This ensures that the push rods 104 are always at the correct height for the device to engage to.

FIG. 18 shows the connection bosses 105 located at the bottom of the push rods 104 in greater detail. The connection bosses 105 are threaded on to the ends of the push rods 104. During assembly the connection bosses 105 are adjusted to the correct overall length and are locked in place by one or more lock nuts 117. The end of the connection bosses 105 have a tapered detail 118 which facilitates engagement of the connection bosses 105 to a lift mechanism of the device. A lower flange 119 of the connection bosses 105 allows a clamp mechanism in the device to clamp down on the connection bosses 105 to ensure that the face 120 of the connection boss 105 is pressed against a seal.

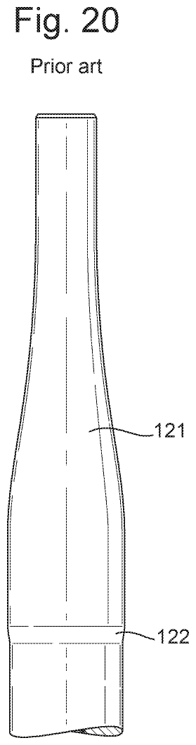

FIGS. 19 and 20 show the upper end 111 of the push rods 104 in greater detail. The push rods 104 may comprise stainless steel for strength, wear resistance and corrosion resistance. The push rods 104 may have a screw-on push rod end 111 which comprises stainless steel and may be titanium nitride coated for wear resistance. The shape of the upper rod ends 111 allows the upper end 111 of the push rods 104 to pass past beads within a cartridge 101 without damaging the beads. The push rod 104 may have ends which are slightly larger in diameter than the diameter of the rest of the push rods 104. This prevents the push rods 104 from sliding through the bearing bushes 108. At the base of the push rod ends 111 a curve 121 may be provided together with a curved end 122 to ensure that reagent beads are not trapped and/or damaged when the push rods 104 are fully retracted.

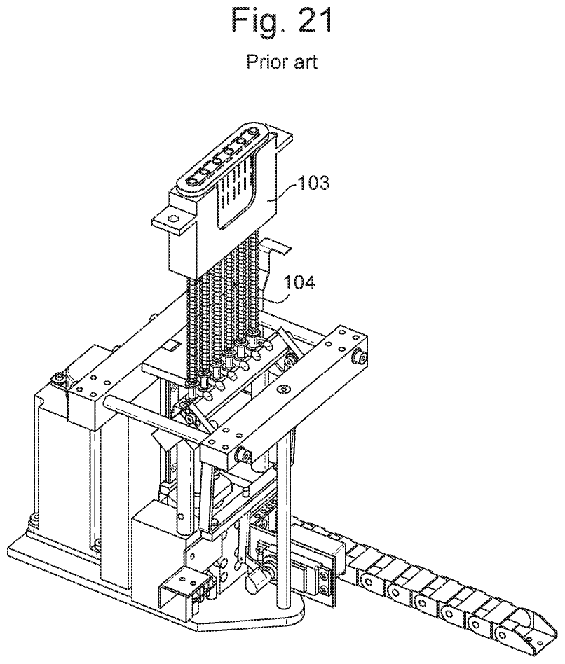

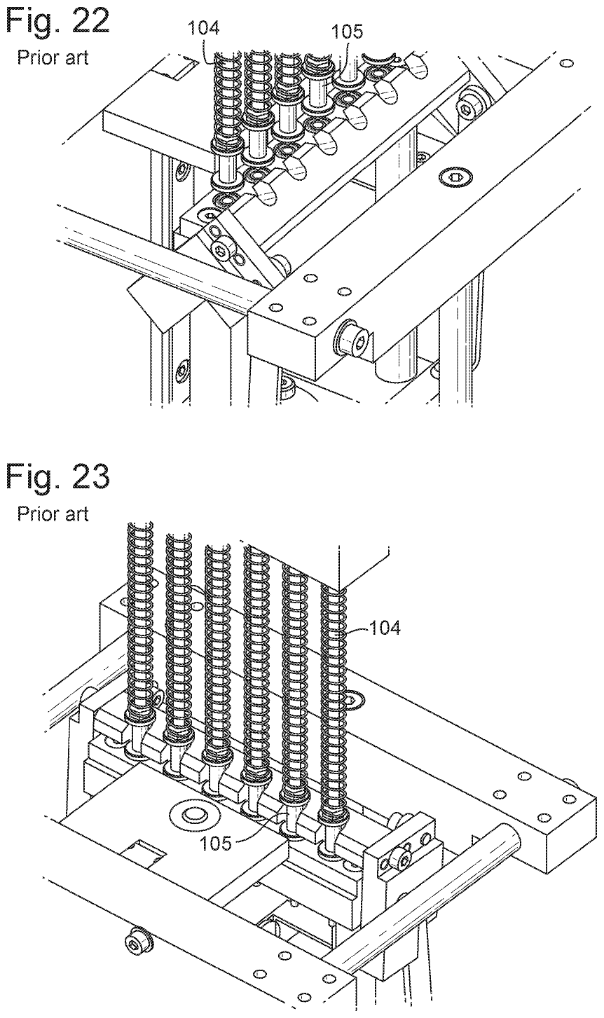

FIG. 21 shows a cartridge holder 103 engaged with a lift mechanism. The lift mechanism may comprise a clamp mechanism which engages with the push rods 104. FIGS. 22 and 23 shows in more detail the lift mechanism being rotated into position so as to engage with the connection bosses 105 provided at the lower end of the push rods 104. FIG. 22 shows the connection bosses 105 in a position where they are not yet clamped to the lift mechanism. FIG. 23 shows the connection bosses 105 engaged with the lift mechanism.

### Cylindrical Beads

According to a particularly preferred embodiment a substantially cylindrical bead design may be used as an alternative to using spherical reagent beads.

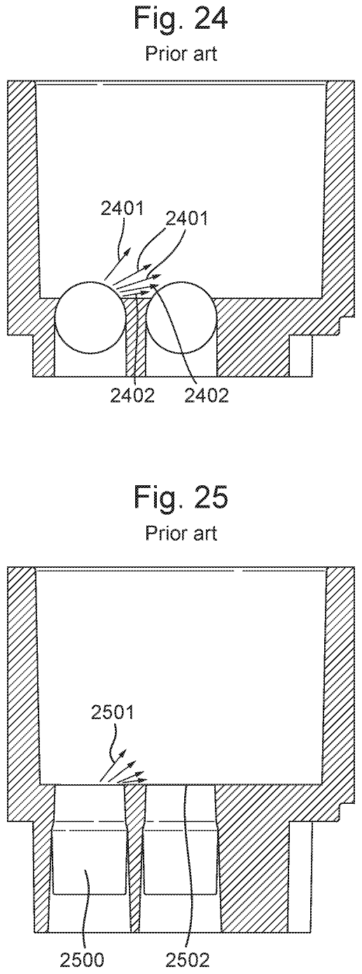

One issue with the known sample plate and the use of spherical reagent beads is that the spherical reagent beads protrude above the base portion of the sample well into the sample well as shown in FIG. 24. As a result, the spherical reagent beads when illuminated (in order to determine the intensity associated with a reagent bead) can emit stray light 2401 or cause light to be reflected onto one or more neighbouring spherical reagent bead(s) causing cross talk. The stray light can hit the surface of other reagent beads at the locations shown by 2402 in FIG. 24. The effect of light 2401 from one reagent bead being reflected onto one or more neighbouring reagent beads adds unwanted light signal to the neighbouring reagent beads in the sample well. It will be appreciated from FIG. 24 that the entire visible surface of a spherical reagent bead which protrudes into the bottom of a sample well will emit light 2401 in essentially a spherical pattern as partly illustrated. Some of the light 2401 which is reflected from one reagent bead onto a neighbouring reagent bead will shine directly on to the non-horizontal face 2402 of the neighbouring spherical reagent beads located within the same sample well. This additional signal on the beads is disadvantageously included in the overall signal from or for a particular reagent bead.

The effect of the unwanted stray light 2401 can be reduced or otherwise mitigated using a software algorithm. However, it will be appreciated that simplifying the process and avoiding any need to use a software algorithm to negate the effects of crosstalk would be advantageous.

The use of substantially cylindrical reagent beads, plugs or inserts according to a particularly preferred embodiment in order to reduce crosstalk and other disadvantageous effects will now be described in more detail with reference to FIG. 25.

FIG. 25 shows an embodiment wherein substantially cylindrical reagent beads, plugs or inserts 2500 having a substantially flat top or substantially flat upper surface are fitted into sample wells of a sample plate such that the top or upper surface of the cylindrical reagent beads, plugs or inserts 2500 is substantially level or flush with the bottom of the fluid surface of the sample well. Accordingly, the substantially cylindrical reagent beads, plugs or inserts 2500 do not substantially protrude into the interior space of the sample well.

A particularly advantageous feature of using substantially cylindrical reagent beads, plugs or inserts 2500 according to a preferred embodiment is that any stray or reflected light 2501 which may be emitted or reflected from the surface of a cylindrical reagent bead, plug or insert 2500 does not shine directly on to or impinge upon a neighbouring cylindrical reagent bead, plug or insert 2502 since the upper surfaces of the substantially cylindrical beads, plugs or inserts lie in substantially the same plane. The substantially cylindrical reagent beads, plugs or inserts 2500 preferably seal into the bore or through hole of the sample well in a similar manner to conventional spherical beads. As a result, a liquid tight seal is preferably formed wherein the substantially cylindrical reagent beads, plugs or inserts 2500 are pressed into the bore or through hole in the sample well and preferably seal against the inner surface of the bore or through hole by way of an interference fit. The seal between the substantially cylindrical reagent bead, insert or plug 2500 and the wall of the bore or through hole is preferably substantially fluid tight so that fluid is preferably prevented from passing beyond or around the fluid tight seal.

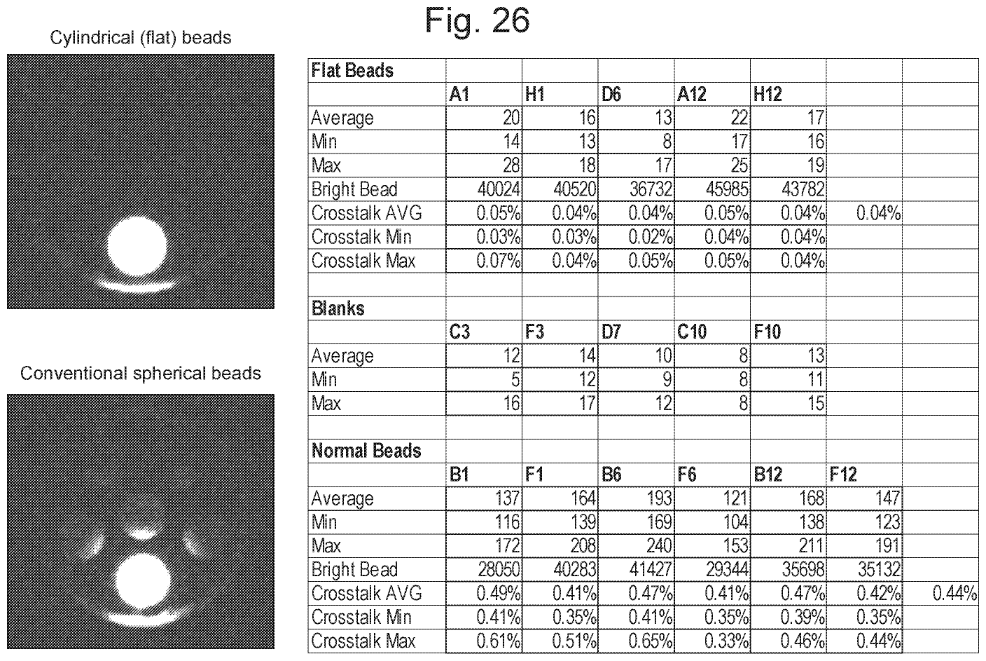

FIG. 26 shows the results of an experiment which was conducted to illustrate how substantially cylindrical reagent beads, plugs or inserts 2500 according to the preferred embodiment having a flat upper surface are particularly effective in substantially reducing and/or effectively eliminating cross talk. A sample well containing five blank beads and one bright bead was imaged using both conventional spherical beads and also flat topped substantially cylindrical beads 2500 according to a preferred embodiment. The intensity values from the five blank beads were read and the results from each well were compared.

The results are shown in FIG. 26 and show that conventional spherical beads pick up approximately 0.44% stray light whereas substantially cylindrical flat reagent beads 2500 according to the preferred embodiment pick up only approximately 0.04% of stray light.

Bead Manufacture Improvement with Cylindrical Beads

Conventional spherical reagent beads or microbeads are manufactured using a grinding process to achieve a uniform finish. In order to ensure that the beads form a liquid tight seal the finish must be kept below a certain level of roughness i.e. the finished reagents beads or microbeads must have a high degree of smoothness.

Table 1 below details some different categories of surface finish and associated roughness.

TABLE 1
roughness

| Roughness | Roughness | |
|---|---|---|
| values Ra | values Ra | Roughness |
| micrometers | micro inches | grade number |
| 50 | 2000 | N12 |
| 25 | 1000 | N11 |
| 12.5 | 500 | N10 |
| 6.3 | 250 | N9 |
| 3.2 | 125 | N8 |
| 1.6 | 63 | N7 |
| 0.8 | 32 | N6 |
| 0.4 | 16 | N5 |
| 0.2 | 8 | N4 |
| 0.1 | 4 | N3 |
| 0.05 | 2 | N2 |
| 0.025 | 1 | N1 |

It is not possible to produce conventional spherical reagent beads or microbeads on a commercial basis using an injection moulding process since an injection moulding process leaves a seam where the parting line is. Furthermore, the injection moulding process also leaves a sprue mark where the plastic was injected.

In contrast to the grinding process which is used to manufacture conventional spherical reagent beads or microbeads, according to a preferred embodiment non-spherical or substantially cylindrical beads, plugs or inserts 2500 can advantageously be manufactured using an injection moulding process. One advantage of using an injection moulding process is that an injection moulding process allows a smooth finish to be formed on the sealing faces (i.e. curved sidewall face) and also an optimal binding finish on the ends (i.e. upper and lower circular faces or surfaces) to be formed. Preferred substantially cylindrical reagent beads, plugs or inserts 2500 manufactured using an injection moulding process therefore enable reagent beads, plugs or inserts 2500 to be provided having good sealing properties wherein the sealing properties are independent from the end face properties. This allows flexibility for the finish on the end face(s) or upper/lower surfaces such that different finishes can be made to suit the assay that the beads are used for.

According to an embodiment an injection mould tool may be used which has textured cavity ends to form the desired finish on the end(s) of the non-spherical or substantially cylindrical reagent beads, plugs or inserts 2500. Accordingly, a desired finish on the end(s) of the non-spherical or substantially cylindrical reagent beads, plugs or inserts 2500 can be produced uniformly across all cavities and is preferably consistent over each moulding cycle giving a high level of bead to bead and lot to lot consistency.

An injection moulding process is commonly used to manufacture standard microtiter plates. An important benefit of using injection moulding is that the end product is less likely to be contaminated by the manufacturing process. Conventional reagent microbeads which are produced using a grinding process are produced using a process which requires a fluid to wash away the ground off material and to prevent clogging. The fluid which is used in the grinding process can act as a source of contamination leading to contaminated beads.

Advantageously, an injection moulding process which is preferably used according to a preferred embodiment is such that only raw resin material comes into contact with the injection mould tool and press. As a result, both the resin material and injection mould tool and press can be simply controlled in order to avoid contamination.

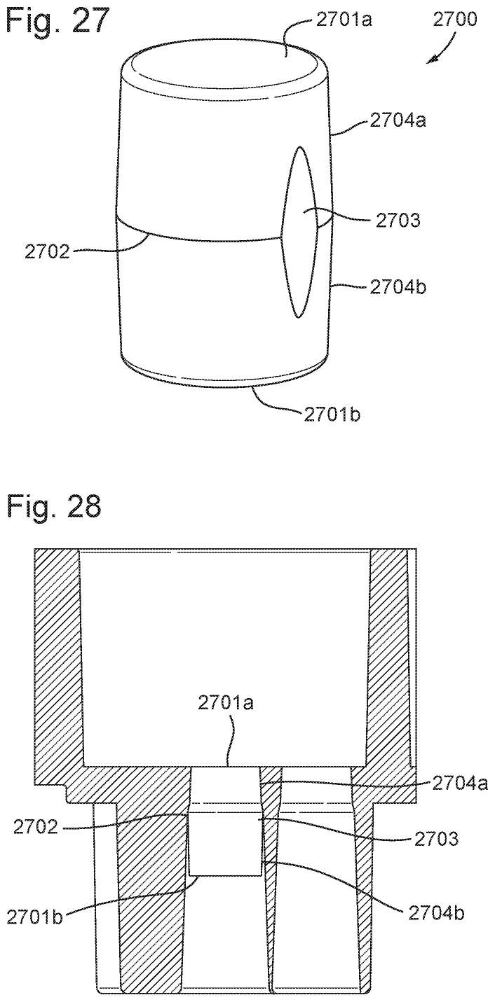

A preferred substantially cylindrical reagent bead, plug or insert 2700 manufactured using an injection moulding process according to a preferred embodiment is shown in FIG. 27. The preferred reagent bead, plug or insert 2700 as shown in FIG. 27 has a first or upper end face or surface 2701a and a second or lower end face or surface 2701b. The preferred substantially cylindrical reagent bead, plug or insert 2700 may have a seam 2702 resulting from the injection moulding process and an imperfection or sprue mark 2703. The reagent bead, plug or insert 2700 preferably has an upper sealing face or surface 2704a and a lower sealing face or surface 2704b.

The preferred reagent bead, plug or insert 2700 preferably provides the following features: (i) a smooth sidewall surface for sealing into the well pockets; (ii) end faces or surfaces 2701a,2701b which may have an optimal textured finish for binding of a reagent; (iii) seam 2702 and sprue 2703 positions which preferably do not affect either the sealing or the end finish of the end faces or surfaces 2701a,2701b; and (iv) optionally a symmetrical design such that the reagent bead, plug or insert 2700 can be fitted either way around into a borehole or through hole of a sample well.

When a substantially cylindrical reagent bead 2700 according to a preferred embodiment is fitted into a sample well of a preferred sample plate the substantially cylindrical reagent bead 2700 preferably seals in the bore, aperture, hole or recess of the sample well by way of an interference fit. The upper end face or surface 2701a of the reagent bead, plug or insert 2700 may be arranged so as to be positioned substantially flush with the bottom of the sample well.

FIG. 28 shows a preferred reagent bead, plug or insert 2700 which is preferably positioned in a bore, aperture, hole or recess of a sample plate such that the seam 2702 and the sprue mark 2703 do not interfere with the sealing performance which is preferably effected by the upper sidewall sealing face 2704a.

### Improvements of Bead to Well Assembly Obtained Using a Stepped Bead Design

Conventional spherical reagent beads and substantially cylindrical reagent beads, plugs or inserts 2700 according to a preferred embodiment both rely upon precise insertion of the reagent bead in order to ensure that the reagent bead is positioned at a precise or desired height, position or depth within the sample well. In the case of preferred substantially cylindrical reagent beads, plugs or inserts 2700 it is necessary to ensure that the preferred substantially cylindrical reagent beads, plugs or inserts 2700 are inserted into holes, apertures or recesses provided in the base portion of a sample well such that a first or upper surface 2701a of the reagent beads, plugs or inserts does not substantially protrude above or beyond the upper surface of the base portion. However, the requirement to position either spherical conventional reagent beads or preferred substantially cylindrical reagent beads, plugs or inserts 2700 at precise positions, locations or heights within a hole or aperture provided in the base portion of a sample well may require the use of a relatively complex robotic bead insertion device. The requirement to use a relatively complex robotic bead insertion device can increase the overall manufacturing cost (or end user cost).

According to a further preferred embodiment as shown in FIG. 29 a reagent bead, plug or insert 2900 according to a preferred embodiment may be provided which is designed so that the height, position or depth of the reagent bead, plug or insert 2900 in the sample well is set by a feature 2901 on the reagent bead, plug or insert 2900. This feature 2901 can be precisely controlled by the injection moulding process when manufacturing the reagent bead, plug or insert 2900.

FIG. 29 shows how a stepped reagent bead, plug or insert 2900 according to a preferred embodiment may be provided having a step feature 2901 that sets or otherwise determines the assembled height, position or depth of a reagent bead, plug or insert 2900 in the base of the sample well.

The stepped bead 2900 may have end faces 2902 having an optimal texture for assay performance. The stepped bead 2900 may have a smooth cylindrical sidewall for sealing into the well and a step feature 2901 to control the insertion height, position or depth. The stepped bead 2900 is preferably symmetrical and the end faces 2902 and side sealing face 2903a,2903b are preferably identical such that the bead, plug or insert 2900 can be inserted either way around into a hole, aperture or recess provided in a sample well of sample plate.

A bead insertion device may be used to a set force in order to insert one or more generally cylindrical reagent beads, plugs or inserts 2900 having a step feature 2901 into a hole, aperture or recess provided in a sample well of a sample plate such that the generally cylindrical reagent beads, plugs or inserts 2900 stop when the step 2901 of the reagent bead, plug or insert 2900 hits a corresponding horizontal face in the well pocket. The insertion device may use simple spring force to insert the beads, plugs or inserts 2900 and may not need to rely on precise positioning of the insertion end piece.

### Crosstalk Reduction Using Flanged Bead Pockets

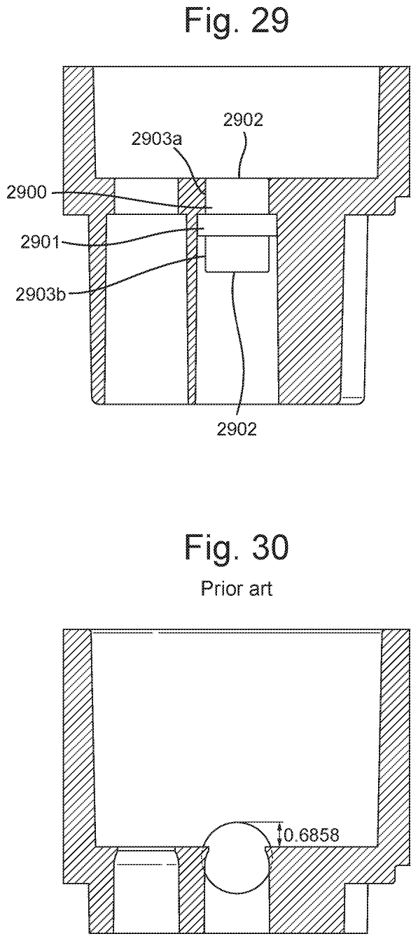

According to the various known arrangements which utilise conventional spherical reagent beads, spherical reagent beads may be pressed in to a though hole of a sample well to a height such that the top of the bead is 0.6858 mm (0.027″) above the bottom of the sample well as shown in FIG. 30. The height of the spherical reagent bead was initially set at 0.50 mm but it was found that the assay precision was improved if the reagent beads extended further above the base portion of the sample well. Beads protruding higher into the well have more contact with fluid in the sample well and this results in a more even reaction.

However, as discussed above, a drawback of having the reagent beads extend higher into the sample well is that more of the reagent beads are then exposed creating more crosstalk within the sample well.

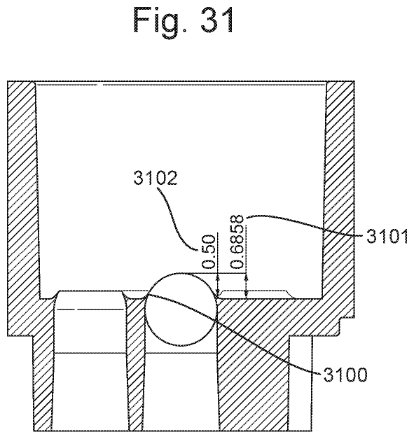

According to an embodiment as shown in FIG. 31 spherical reagent beads may be used wherein an additional flange, rim, collar or raised portion 3100 is moulded into or otherwise formed in the base portion of one or more sample wells allowing the spherical reagent beads to remain at a bead height 3101 of 0.6858 mm above the base of the sample well so as to retain the same amount of contact with fluid in the sample well but wherein the flange, rim, collar or raised portion 3100 blocks a proportion of stray light at the lower part of the reagent bead leaving 0.5 mm of the reagent bead still exposed to sample fluid i.e. the reagent bead has an exposed height 3102 of 0.5 mm.

### Tapered Cylindrical Reagent Beads or Inserts

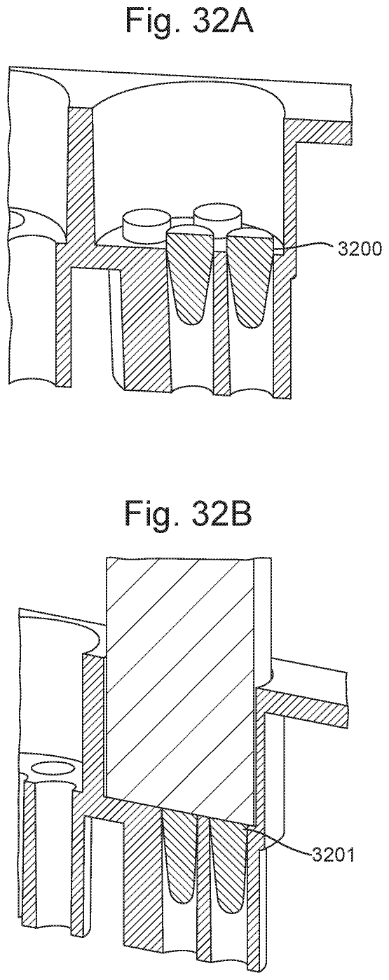

Conventional spherical reagent beads and substantially cylindrical reagent beads according to a preferred embodiment both rely upon precise insertion of the reagent bead in order to ensure that the beads are positioned at a precise height within the sample well. This can increase the complexity and hence the cost of associated bead insertion equipment. According to an embodiment as shown in FIG. 32A a tapered bead may be provided which can be inserted from the top (as opposed to via the underneath of the sample plate as in the case with reagent beads, plugs or inserts 2900 having a step feature 2901 as shown in FIG. 29).

According to an embodiment an automated bead insertion device may be provided wherein tapered reagent beads, plugs or inserts are initially dropped or partially inserted into bead bores, holes or apertures in the sample wells and wherein the reagent beads, plugs or inserts are then collectively pressed into place using a press-in tool. The reagent beads, plugs or inserts are preferably pressed in so that the beads, plugs or inserts are preferably flush with the bottom of the well eliminating the need for precise insertion methods. FIG. 32A shows tapered reagent beads, plugs or inserts according to an embodiment after being dropped or partially inserted into the well pockets or recesses and shows the reagent beads, plugs or inserts dropped in or inserted to a certain height above the base of the sample well. FIG. 32B shows a press-in tool pressing the tapered reagent beads, plugs or inserts fully into place according to an embodiment wherein the beads, plugs or inserts are pressed-in flush 3201 with the bottom of the sample well.

A flat ended press-in tool as shown in FIG. 32B may according to an embodiment be used to make contact with the entire top face of the tapered beads, plugs or inserts thereby preventing any damage to any reagent or other coating on the reagent beads, plugs or inserts. Since different bead, plug or insert types for a given assay preferably always go into the same position, the part of the press-in tool that contacts the top of the reagent beads, plugs or inserts can remain constant thereby avoiding any cross contamination caused by the process of inserting the reagent beads, plugs or inserts.

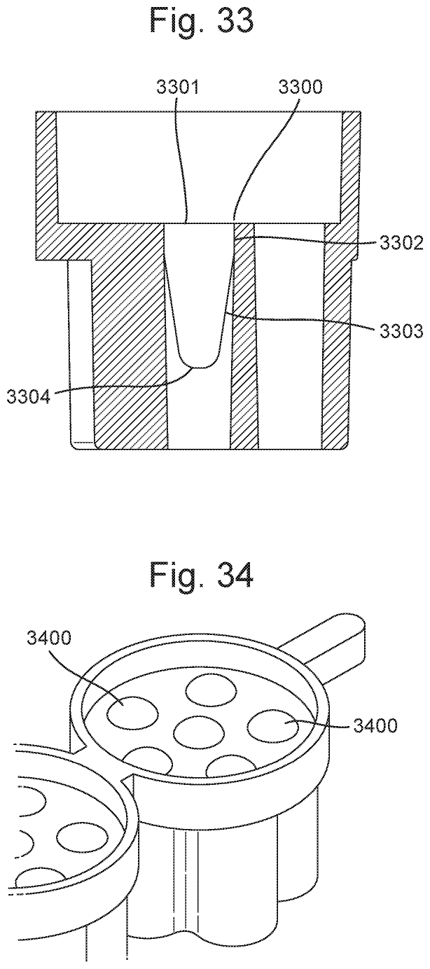

The tapered reagent beads may according to an embodiment be arranged to have a square edge 3300 to the top as shown in FIG. 33 so that when pressed the reagent beads, plugs or inserts do not create a fluid trap around the circumference of the reagent bead, plug or insert. The tapered reagent beads, plugs or inserts can be injection moulded with the sprue mark located towards the tapered end so that the sprue mark does not affect the performance of the reagent bead, plug or insert when the reagent bead, plug or insert is inserted into a hole, aperture or recess in the base of the sample well. The upper end face or surface 3301 of the reagent bead, plug or insert may have a different finish to the sides. For example, according to an embodiment a smooth surface may be provided on the sides or side sealing face 3302 to provide a good sealing. The tapered reagent bead, plug or insert may have a relatively rough end face 3301 which may be more suitable for assay performance. The end face 3301 may have a roughness as indicated in Table 1 above. The reagent beads, plugs or inserts may taper 3303 to ease assembly and may have a radius 3304 at one end to ease assembly.

### Assay Performance Improvement Using Either Cylindrical or Stepped Beads

During an assay process the sample wells may be agitated (i.e. shaken) in order to ensure that the sample fluid moves around within the bottom of the sample well so as to provide an even distribution of the fluid molecules over the reagent beads, plugs or inserts. With the conventional arrangement as show in FIG. 30 wherein spherical beads protrude into the bottom of the sample wells, the internal shape or profile of the bottom of the sample wells is non-flat as shown in FIG. 34 due to the spherical beads 3400 protruding into the sample well.

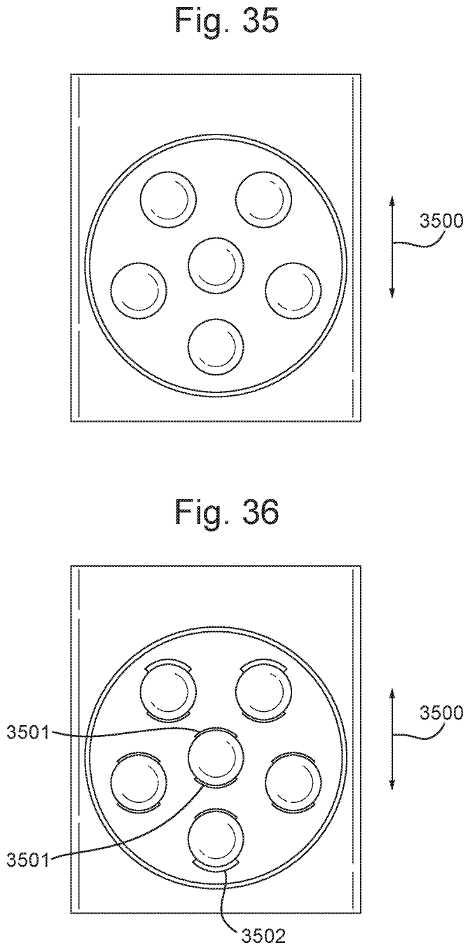

If a linear shake is used or performed then fluid in the base of a sample well moves back and forth along the direction of the shake 3500 as shown in FIG. 35.

Although the spherical beads produce a non-flat shape or profile in the bottom of the well, it is still uniform and consistent on all wells. A linear shake will produce a pattern over time due to the repetition of fluid flow such that the amount of fluid flow over each bead will be different leading to a variance in the end result depending on the position of a reagent bead.

With spherical beads protruding into the bottom of a sample well the fluid flow is interrupted which can create areas where the fluid does not flow (i.e. dead zones). The creation of dead zones creates less transfer of molecules from the fluid on to the reagent bead causing a reduction in signal compared to areas where the fluid does flow.

FIG. 36 shows an example of how spherical beads which protrude into a sample well produce fluid dead zones and wherein these zones will differ depending on where the bead is. As illustrated in FIG. 36 a centrally located reagent bead will create a smaller dead zone 3501 relative to a reagent bead located around the circumference of the base portion of the sample well which will create a larger dead zone 3502.

By way of contrast, the cylindrical or stepped beads, plugs or inserts according to various preferred embodiments as described above preferably do not protrude beyond the base of the sample wells into the sample well. According to a preferred embodiment the base of a sample well is therefore substantially flat or planar without portions of the reagent beads, plugs or inserts projecting above the base of the sample well. Advantageously, the fluid flow is therefore not interrupted. As a result, fluid flow dead zones are substantially prevented from forming. This advantageously results in a more uniform transfer of molecules from the fluid to the reagent beads, plugs or inserts irrespective of the position of the reagent beads, plugs or inserts.

It will be apparent, therefore, that the use of non-spherical reagent beads, plugs or inserts according to the preferred embodiments represents a significant advance in the art.

Although the present invention has been described with reference to preferred embodiments, it will be understood by those skilled in the art that various changes in form and detail may be made without departing from the scope of the invention as set forth in the accompanying claims.

1-82. (canceled)

83. A kit comprising:
one or more non-spherical reagent beads, plugs or inserts; and
a sample plate comprising one or more sample wells, wherein one or more of the sample wells comprise:
a base portion having an upper surface which forms a bottom portion of the sample well; and
one or more holes or apertures provided in the base portion;
wherein one or more of the non-spherical reagent beads, plugs or inserts are substantially retained or secured, in use, within the one or more holes or apertures so as to form a substantially fluid-tight circumferential seal with a wall of the base portion which defines the hole or aperture and wherein the upper surface of the one or more reagent beads, plugs or inserts is substantially flush with or co-planar with the upper surface of the base portion so that an upper surface of the one or more reagent beads, plugs or inserts does not substantially protrude above or beyond the upper surface of the base portion.

84. A kit as claimed in claim 83, wherein the one or more reagent beads, plugs or inserts comprise one or more substantially or generally cylindrical reagent beads, plugs or inserts.

85. A kit as claimed in claim 83, wherein the one or more reagent beads, plugs or inserts have a substantially or generally circular, round, oval, curved, square, rectangular, polygonal, regular or irregular cross-sectional profile.

86. A kit as claimed in claim 83, wherein the one or more reagent beads, plugs or inserts comprise one or more substantially prism shaped or prismatic reagent beads, plugs or inserts.

87. A kit as claimed in claim 83, wherein the one or more reagent beads, plugs or inserts have a cross-sectional profile which either: (i) remains substantially constant along the full longitudinal length of the reagent bead, plug or insert; or (ii) varies, changes or tapers one or more portions of the longitudinal length of the reagent bead, plug or insert.

88. A kit as claimed in claim 83, wherein the one or more reagent beads, plugs or inserts have a substantially or generally circular cross-sectional profile and wherein the diameter of the one or more reagent beads, plugs or inserts in a middle portion of the reagent beads, plugs or inserts is greater than at one or both end portions of the reagent beads, plugs or inserts.

89. A kit as claimed in claim 83, wherein the one or more reagent beads, plugs or inserts have a substantially or generally circular cross-sectional profile and wherein the diameter of the one or more reagent beads, plugs or inserts tapers or narrows towards one or both end portions of the reagent beads, plugs or inserts.

90. A kit as claimed in claim 83, wherein the one or more reagent beads, plugs or inserts have a first end face, wherein the first end face is coated with a reagent or includes a reagent.

91. A kit as claimed in claim 90, wherein the one or more reagent beads, plugs or inserts have a second end face, wherein the second end face is also coated with or includes a reagent.

92. A sample plate comprising one or more sample wells, wherein one or more of the sample wells comprise:
a base portion having an upper surface which forms a bottom portion of the sample well;
one or more holes or apertures provided in the base portion; and
one or more raised portions, flanges, rims or collars surrounding the one or more holes or apertures;
wherein one or more reagent beads, plugs or inserts are substantially retained or secured, in use, within the one or more holes or apertures so as to form a substantially fluid-tight circumferential seal with either a wall of the base portion which defines the hole or aperture and/or the one or more raised portions, flanges rims or collars.

93. A sample plate as claimed in claim 92, wherein the one or more reagent beads, plugs or inserts are substantially or generally spherical.

94. A sample plate as claimed in claim 92, wherein the one or more holes or apertures comprise one or more open through holes.

95. A sample plate as claimed in claim 92, wherein the one or more holes or apertures are substantially or generally cylindrical.

96. A sample plate as claimed in claim 92, wherein the one or more holes or apertures have a cross-sectional profile which either: (i) remains substantially constant along the full longitudinal length of the hole or aperture; or (ii) varies, changes or tapers along one or more portions of the longitudinal length of the hole or aperture.

97. A sample plate as claimed in claim 92, wherein the one or more holes or apertures have a diameter less than a diameter of a reagent bead, plug or insert deposited in the hole or aperture so that the reagent bead, plug or insert is retained or secured within the hole or aperture by an interference or friction fit.

98. A reagent bead, plug or insert for use with the kit of claim 83.

99. The reagent bead, plug or insert of claim 98, having a first end face, wherein the first end face is coated with a reagent or includes a reagent.

100. The reagent bead, plug or insert of claim 98, having a circumferential step portion, flange or stopper feature.

101. The reagent bead, plug or insert of claim 98, having a square upper edge or an edge which in use abuts substantially parallel to or flush with a corresponding surface of the base portion which defines the one or more holes or apertures.

102. The reagent bead, plug or insert of claim 98, wherein the reagent bead, plug or insert is formed by an injection moulding process.
